(19) United States
(12) Patent Application Publication
(10) Pub. No.: US 2015/0168428 A1
(43) Pub. Date: Jun. 18, 2015

(54) COMPOSITIONS AND METHODS FOR ASSESSING CARDIOVASCULAR DISEASE

(71) Applicant: President and Fellows of Harvard College, Cambridge, MA (US)

(72) Inventors: Frank M. Sacks, Belmont, MA (US); Eric Rimm, Brookline, MA (US); Majken Jensen, Brookline, MA (US); Jeremy D. Furtado, Berkley, MA (US)

(21) Appl. No.: 14/399,763

(22) PCT Filed: May 9, 2013
(86) PCT No.: PCT/US13/40430

§ 371 Date: Nov. 7, 2014

Related U.S. Application Data
(60) Provisional application No. 61/644,901, filed on May 9, 2012.
(60) Provisional application No. 61/659,576, filed on Jun. 14, 2012.
(60) Provisional application No. 61/798,575, filed on Mar. 15, 2013.

(51) Int. Cl. G01N 33/92

(52) U.S. Cl. CPC G01N 33/92; G01N 2800/32; G01N 2800/50; G01N 2333/775; G01N 2800/324; G01N 2800/323; G01N 2800/52

(57) ABSTRACT

Some aspects of this disclosure relate to the characterization of lipoproteins (e.g., vLDL, LDL, and/or HDL) based on their content of lipoprotein-associated proteins (e.g., apolipoproteins) for the determination of cardiovascular disease risk. Some aspects of this disclosure relate to methods for the diagnosis, early detection, risk estimation and monitoring of the course of diseases, in which one or more lipoprotein-associated protein is detected in a lipoprotein. The characterization of the levels of lipoproteins with different lipoprotein-associated protein content provide an index for assessing the risk of a disease, for example, a cardiovascular disease.

### RELATED APPLICATIONS

This application claims the benefit of the filing date of priority to U.S. Provisional Application Ser. No. 61/644,901, filed on May 9, 2012, U.S. Provisional Application Ser. No. 61/659,576, filed on Jun. 14, 2012, and U.S. Provisional Application Ser. No. 61/798,575, filed on Mar. 15, 2013, the contents of all of which are incorporated by reference in their entirety.

### BACKGROUND

Lipoproteins are lipid-protein complexes that transport lipids (e.g., cholesterol, triglycerides) and other hydrophobic compounds within the circulation of the body. The main lipoproteins that circulate in blood are VLDL, LDL, and HDL. Traditionally, high levels of VLDL and LDL are believed to be associated with an increased risk of developing coronary heart disease (CHD), whereas high levels of HDL are believed to be associated with reduced CHD risk. Clinically, CHD risk is determined by the cholesterol content of these lipoproteins, or by the plasma total triglycerides (TG) levels. However, the assessment of CHD risk based on VLDL, LDL, and HDL measurements alone does not accurately predict much of the incidence of CHD in the population. For example, some patients who have CHD have high blood levels of HDL- cholesterol, and some people who never get CHD have low HDL-cholesterol levels. A better understanding of CHD risk factors is needed.

### SUMMARY

Some aspects of this disclosure provide methods of generating an index for assessing the risk of a subject for having or developing cardiovascular disease. In some embodiments, the method comprises (i) detecting an analyte that is a component of a lipoprotein in a biological sample derived from the subject; and (ii) generating an index that is a measure of the risk of the subject for having or developing cardiovascular disease.

In some embodiments, detecting the analyte comprises measuring the presence or a level or concentration of the analyte. In some embodiments, the level of the analyte is measured via a quantitative or semi-quantitative assay.

In some embodiments, generating the index comprises calculating an index score based on the detection and/or quantification of the analyte.

In some embodiments, the index generated is an index selected from the group consisting of VLDL-LDL Atherogenicity Index, HDL Protection Index, Global Lipoprotein Index, and any combination thereof.

Some aspects of this disclosure provide methods of generating an index for assessing risk of a subject having or developing a cardiovascular disease wherein the index is selected from the group consisting of VLDL-LDL Atherogenicity Index, HDL Protection Index, Global Lipoprotein Index, and any combination thereof, the method comprising detecting at least one analyte that is a component of a lipoprotein in a biological sample derived from the subject and determining the concentration of the analyte in the sample to generate an index that is a measure of the risk of the subject for having or developing a cardiovascular disease.

In some embodiments, when the VLDL-LDL Atherogenicity Index value is higher than a control value indicating low risk, there is an increased risk of cardiovascular disease in the subject.

In some embodiments, when the HDL Protection Index value is lower than a control value denoting low risk, there is an increased risk of cardiovascular disease in the subject.

In some embodiments, the VLDL-LDL Atherogenicity Index value is calculated as the sum of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, when the analyte is associated directly with high risk of cardiovascular disease, a score of 0 is assigned to subjects whose concentrations lie within the first quintile (lowest 20^{th} percentile), a score of 1 is assigned to subjects whose concentrations lie within the second quintile, a score of 2 is assigned to subjects whose concentrations lie within third quintile, a score of 3 is assigned to subjects whose concentrations lie within fourth quintile, and a score of 4 is assigned to subjects whose concentrations lie within the fifth quintile (highest 20^{th} percentile).

In some embodiments, the analyte associated with cardiovascular disease is selected from the group consisting of apoC-II, apoC-III, and any combination thereof.

In some embodiments, the analyte is associated with protection against cardiovascular disease, a score of 4 is assigned to subjects whose concentrations lie within the first quintile (lowest 20^{th} percentile), a score of 3 assigned to subjects whose concentrations lie within the second quintile, a score of 2 assigned to subjects whose concentrations lie within third quintile, a score of 1 assigned to subjects whose concentrations lie within fourth quintile, and a score of 0 assigned to subjects whose concentrations lie within the fifth quintile (highest 20^{th} percentile).

In some embodiments, the analyte associated with protection against cardiovascular disease is apoE.

In some embodiments, the VLDL-LDL Atherogenicity Index value is calculated as the sum of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the VLDL-LDL Atherogenicity Index value is calculated as the multiplication of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the analyte is associated directly with cardiovascular disease, a score of 1 is assigned to subjects whose concentrations lie within the first quintile (lowest 20^{th} percentile), a score of 2 is assigned to subjects whose concentrations lie within the second quintile, a score of 3 is assigned to subjects whose concentrations lie within third quintile, a score of 4 is assigned to subjects whose concentrations lie within fourth quintile, and a score of 5 is assigned to subjects whose concentrations lie within the fifth quintile (highest 20^{th} percentile).

In some embodiments, the analyte associated with cardiovascular disease is selected from the group consisting of apoC-II, apoC-III, and any combination thereof.

In some embodiments, when the analyte is associated with protection against cardiovascular disease, a score of 1 is assigned to subjects whose concentrations lie within the first quintile (lowest 20^{th} percentile), a score of 0.8 assigned to subjects whose concentrations lie within the second quintile, a score of 0.6 assigned to subjects whose concentrations lie within third quintile, a score of 0.4 assigned to subjects whose concentrations lie within fourth quintile, and a score of 0.2 assigned to subjects whose concentrations lie within the fifth quintile (highest 20^{th} percentile). Alternatively, the scores may be simply relative risks.

In some embodiments, the analyte associated with protection against cardiovascular disease is apoE.

In some embodiments, the VLDL-LDL Atherogenicity Index value is calculated as the multiplication of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the VLDL-LDL Atherogenicity Index value is calculated as the sum of scores of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the score is calculated as the product of the coefficient from the linear regression of the analyte on CHD risk and the concentration of the analyte.

In some embodiments, the analyte is selected from the group consisting of apoC-II, apoC-III, and apoE in apoB-lipoproteins, and any combination thereof.

In some embodiments, the HDL Protection Index value is calculated as the summation of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the analyte is associated directly with a higher rate of cardiovascular disease, a score of 4 is assigned to subjects whose concentrations lie within the first quintile (lowest 20^{th} percentile), a score of 3 is assigned to subjects whose concentrations lie within the second quintile, a score of 2 is assigned to subjects whose concentrations lie within third quintile, a score of 1 is assigned to subjects whose concentrations lie within fourth quintile, and a score of 0 is assigned to subjects whose concentrations lie within the fifth quintile (highest 20^{th} percentile).

In some embodiments, the analyte associated with cardiovascular disease is apoC-III in HDL and apoE in HDL.

In some embodiments, when the analyte is associated with protection against cardiovascular disease, a score of 0 assigned to subjects whose concentrations lie within the first quintile (lowest 20^{th} percentile), a score of 1 assigned to subjects whose concentrations lie within the second quintile, a score of 2 assigned to subjects whose concentrations lie within third quintile, a score of 3 assigned to subjects whose concentrations lie within fourth quintile, and a score of 4 assigned to subjects whose concentrations lie within the fifth quintile (highest 20^{th} percentile).

In some embodiments, the analyte associated with protection against cardiovascular disease is HDL without one or more of apoC-III and apoE.

In some embodiments, the HDL Protection Index value is calculated as the sum of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the HDL Protection Index value is calculated as the multiplication of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, when the analyte is associated directly with cardiovascular disease, a score of 0.2 is assigned to subjects whose concentrations lie within the first quintile (lowest 20^{th} percentile), a score of 0.4 is assigned to subjects whose concentrations lie within the second quintile, a score of 0.6 is assigned to subjects whose concentrations lie within third quintile, a score of 0.8 is assigned to subjects whose concentrations lie within fourth quintile, and a score of 1 is assigned to subjects whose concentrations lie within the fifth quintile (highest 20^{th} percentile).

In some embodiments, the analyte associated with cardiovascular disease is apoC-III in HDL and apoE in HDL.

In some embodiments, the analyte is associated with protection against cardiovascular disease, a score of 1 is assigned to subjects whose concentrations lie within the first quintile (lowest 20^{th} percentile), a score of 2 assigned to subjects whose concentrations lie within the second quintile, a score of 3 assigned to subjects whose concentrations lie within third quintile, a score of 4 assigned to subjects whose concentrations lie within fourth quintile, and a score of 5 assigned to subjects whose concentrations lie within the fifth quintile (highest 20^{th} percentile).

In some embodiments, the analyte associated with protection against cardiovascular disease is HDL without one or more of apoC-III and apoE.

In some embodiments, the HDL Protection Index value is calculated as the multiplication of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the HDL Protection Index value is calculated as the sum of scores of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

In some embodiments, the score is calculated as the product of the coefficient from the linear regression of the analyte on CHD risk and the concentration of the analyte.

In some embodiments, the analyte is selected from the group consisting of apoC-III in HDL, apoE in HDL, apoAI without apoC-III or apoE, and any combination thereof.

In some embodiments, the analyte is selected from the group consisting of an integral apolipoprotein, a non-integral apolipoprotein, a lipoprotein-associated protein listed in Table 1, and any combination thereof.

In some embodiments, the integral apolipoprotein is selected from the group consisting of apoA-I, apoB, and any combination thereof.

In some embodiments, the non-integral apolipoprotein is selected from the group consisting of apoA-II, apoC-I, apoC-II, apoC-III, apoE, and any combination thereof.

In some embodiments, the lipoprotein is selected from the group consisting of VLDL, LDL, HDL, and any combination thereof.

In some embodiments, the lipoprotein is computed as the cholesterol or triglyceride concentration.

Some aspects of this disclosure provide methods of assessing a cardiovascular disease in a subject. In some embodiments, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease.

Some aspects of this disclosure provide methods of selecting a subject for participation in a clinical trial, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to select a subject for participation in a clinical trial.

Some aspects of this disclosure provide methods of assessing the efficacy of a pharmaceutical agent, dietary supplement or food product in preventing or treating a cardiovascular disease in a subject in need thereof, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to assess the efficacy of a pharmaceutical agent in treating a subject in need thereof.

Some aspects of this disclosure provide methods of assessing the efficacy of a therapeutic agent, such as a pharmaceutical agent, dietary supplement or food product in treating a cardiovascular disease in a subject in need thereof, the method comprising generating an index that is itself a target for the pharmaceutical agent, dietary supplement or food product. The index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to assess the efficacy of a therapeutic agent in treating a subject in need thereof. The therapeutic agent seeks to lower the VLDL-LDL Atherogenicity Index, raise the HDL Protection Index, and/or lower the Global Lipoprotein Index, thereby lowering the risk of cardiovascular disease in the subject or group of subjects.

Some aspects of this disclosure provide methods of generating an HDL Protection Index, the method comprising combining at least two indices from the group consisting of a classical apolipoprotein index, a thrombogenic index, an inflammation index, and an anti-oxidant index.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1. ApoC-III is a potential modulator of the association between HDL-cholesterol and incident of coronary heart disease. FIG. 1A depicts a flowchart for the nested prospective case-control studies of apoC-III. FIG. 1B is a table depicting the incidence rate ratios (IRR) and 95% confidence intervals of CHD according to quintiles of total HDL-C, HDL-C without ApoC-III and HDL-C with ApoC-III in the Nurses' Health Study (NHS) and the Health Professional Follow-Up Study (HPFS). Incidence rate ratios (IRR) obtained from conditional logistic regression models. Unadjusted model takes into account age and smoking (due to matching). Multivariate model includes: alcohol, body mass index, self-reported diagnosis of hypertension before blood draw, and postmenopausal status and hormones in NHS only. HDL with and without apoC-III are simultaneously included in all models. P trend is the test for linear trend across quintiles. FIG. 1C is a graph depicting the multivariate-adjusted RRs for CHD according to quintiles of total HDL-C, HDL-C with and without apoC-III in the combined NHS and HPFS. RRs are incidence rate ratios (IRR) obtained from conditional logistic regression models. Multivariate model takes into account age and smoking due to matching. HDL-C with and without apoC-III are simultaneously included. NHS and HPFS data were combined using random effects meta-analyses. No P values for test of between study heterogeneity were lower than 0.5. Error bars indicate 95% confidence interval. Adjusted for alcohol, body mass index, self-reported diagnosis of hypertension before blood draw, postmenopausal status, and hormones in NHS. The P for linear trend across quintiles: total HDL-C=0.005; for HDL-C without apoC-III=0.007; HDL-C with apoC-III=0.02. P for test of difference in slope between HDL-C with and without apoC-III=0.02. FIG. 1D is a table depicting the IRR and 95% confidence intervals of CHD according to continuous measures of total HDL-C (per 0.60 mmol/L), HDL-C without ApoC-III (per 0.53 mmol/L), and HDL-C with ApoC-III (per 0.07 mmol/L) in the Nurses' Health Study (NHS) and the Health Professional Follow-Up Study (HPFS). Incidence rate ratios (IRR) obtained from conditional logistic regression models. Unadjusted model takes into account age and smoking (due to matching). Multivariate model includes: alcohol, body mass index, self-reported diagnosis of hypertension before blood draw, and postmenopausal status and hormones in NHS only. HDL with and without apoC-III are simultaneously included in all models. The NHS and HPFS data were combined using random effects meta-analyses. P het=P for test of between study heterogeneity.

FIG. 2. Relative risks of CHD according to levels of measured apolipoprotein contents and molar ratios in VLDL and LDL, adjusted for different plasma lipids and C-Reactive protein. Each bar represents a different model. FIG. 2A depicts results adjusted for plasma LDL cholesterol. FIG. 2B depicts results adjusted for plasma HDL cholesterol. FIG. 2C depicts results adjusted for plasma triglycerides. FIG. 2D depicts results adjusted for plasma CRP. All models contained were additionally adjusted for matching factors, parental history of CHD before the age of 60 years, personal history of hypertension, alcohol intake, body mass index and personal history of diabetes. Bars represent relative risks for quintile 5 compared to quintile 1 of each variable.

FIG. 3. Baseline characteristics of the study sample. Data on women are from the Nurses' Health Study and include fourteen years of follow-up, and data on men are from the Health Professionals Follow-up Study and include ten years of follow-up. Matching criteria were age, smoking status, and date of blood sampling; among women, additional matching criteria included fasting status at the time of blood sampling. Plus-minus values are means±SD. CHD denotes coronary heart disease. The body mass index is the weight in kilograms divided by the square of the height in meters. ** P values for the difference between patients and controls (unadjusted) were determined by Student's t-test for variables expressed as means±SD, by Wilcoxon's rank-sum test for variables expressed as medians, and by the chi-square test for variables expressed as percentages. *** Current aspirin use was defined as every one to four days per week for women and as two or more times per week for men.

FIG. 4. Relative risks of CHD by quintiles of apoE:apoB and apoE:apoC-III molar ratios in VLDL and LDL. Relative risks and 95% confidence intervals are given for each quintile compared to the lowest quintile of each apolipoprotein ratio. The group of women included 322 cases and 322 controls with fourteen years of follow-up. The group of men included 418 cases and 418 controls with ten years of follow-up. Quintiles and median values of apolipoprotein levels are based on values in controls. For each relative risk, quintile 1 served as the reference group. *Conditioned on matching factors only. Matching factors were age, smoking status, and the month of blood sampling. Among women, data were also adjusted for fasting status at the time of blood sampling. ** Adjusted for matching factors, parental history of CHD before the age of 60 years, alcohol intake and personal history of hypertension. *** Adjusted as in model 2 plus for body mass index and personal history of diabetes, **** P values for trend are based on the median levels of apolipoprotein ratios in quintiles of the controls.

FIG. 5. Relative risk of coronary heart disease during follow-up in the complete study sample, according to levels of apolipoprotein (apo) B in low-density lipoprotein (LDL) with apoC-III, in models including other major lipid risk factors. Each part represents a separate model in which apoB and another lipid risk factor were mutually adjusted. Solid bars represent relative risks for quintile 5 vs. 1 of each risk factor; error bars represent 95% confidence intervals. All models were adjusted for matching factors, presence or absence of a parental history of coronary heart disease before 60 years of age, alcohol intake, and personal history of hypertension. HDL indicates high-density lipoprotein.

FIG. 6. Relative risk of coronary heart disease during follow-up in the complete study sample, mutually adjusting for apolipoprotein (apo) B in low-density lipoprotein (LDL) with and without apoC-III. Relative risks and 95% confidence intervals are given for each quintile vs. the lowest quintile. FIG. 6A is a graph depicting a model which was also adjusted for matching factors, presence or absence of a parental history of coronary heart disease before 60 years of age, alcohol intake, and personal history of hypertension. FIG. 6B is a graph depicting a model, which was adjusted for all variables in FIG. 6A plus personal history of diabetes mellitus and plasma triglycerides. Dark diamonds represent LDL with apoC-III; P for trend <0.001 in FIG. 6A and P for trend=0.07 in FIG. 6B, Light squares represent LDL without apoC-III; P for trend=0.97 in FIG. 6A and P for trend=0.22 in FIG. 6B. P<0.001 for difference in slopes in FIG. 6A and P=0.001 for difference in slopes in FIG. 6B.

FIG. 7. Relative risk of coronary heart disease during follow-up, according to the tertile of apolipoprotein (apo) B in low-density lipoprotein (LDL) with apoC-III and the tertile of apoC-III in LDL at baseline. Subjects in tertile 1 of apoB in LDL with apoC-III and tertile 1 of apoC-III in LDL served as the reference group. The model was also adjusted for matching factors, presence or absence of a parental history of coronary heart disease before 60 years of age, alcohol intake, personal history of hypertension, personal history of diabetes mellitus, and plasma triglycerides.

FIG. 8. Baseline characteristics of the study sample. CHD denotes coronary heart disease; Q1, quartile 1; Q3, quartile 3; LDL, low-density lipoprotein; HDL, high-density lipoprotein; apo, apolipoprotein; and VLDL, very low-density lipoprotein. Plus-minus values are mean_SD. To convert values for cholesterol to milligrams per deciliter, multiply by 38.6. To convert values for triglycerides to milligrams per deciliter, multiply by 88.57. The body mass index is weight in kilograms divided by the square of the height in meters. *Data on women are from the Nurses' Health Study; data on men are from the Health Professionals Follow-up Study. Matching criteria were age, smoking status, and date of blood sampling; among women, additional matching criteria included fasting status at the time of blood sampling. †P values for the difference between cases and controls (unadjusted) were determined by paired Student t test for variables expressed as mean±SD, by the signed-rank test for variables expressed as medians, and by the McNemar χ² test for variables expressed as percentages. ‡Current aspirin use was defined as 1 to 4 d/wk for women and as ≧2 times per week for men.

FIG. 9. Relative risks of coronary heart disease during follow-up in the complete study sample, according to the quintile of low-density lipoprotein types (as measured by the apolipoprotein b concentration in each fraction) or apolipoprotein concentrations at baseline. LDL indicates low-density lipoprotein; apo, apolipoproteins. Relative risks and 95% confidence intervals are given for each quintile compared with the lowest quintile of each apolipoprotein measurement. The group of women included 320 cases and 320 controls with 14 years of follow-up. The group of men included 419 cases and 419 controls with 10 years of follow-up. Quintiles and median values of apolipoprotein levels are based on values in controls. For each relative risk, quintile 1 served as the reference group. Matching factors were age, smoking status, and the month of blood sampling. Among women, data were also adjusted for fasting status at the time of blood sampling.*Model 1 is conditioned only on matching factors. Model 2 is also adjusted for the presence or absence of a parental history of coronary heart disease before 60 years of age, alcohol intake, and personal history of hypertension. Model 3 is adjusted for all variables in model 2 plus body mass index and personal history of diabetes mellitus. Model 4 is adjusted for all variables in model 3 plus plasma triglycerides. †P values for trend are based on the median levels of apolipoproteins in quintiles of the controls. ‡Calculated as the P for the interaction between sex and median apolipoprotein levels in quintiles of the controls.

FIG. 10. Relative risks of coronary heart disease during follow-up, according to the quintile of apolipoprotein concentrations at baseline, by sex. * Model 1 was conditioned on matching factors (age, smoking status, and the month of blood sampling). Among women, data were also conditioned on fasting status at the time of blood sampling. Model 2 was additionally adjusted for presence or absence of a parental history of coronary heart disease before the age of 60 years, alcohol intake and personal history of hypertension. Model 3 was adjusted for all the variables in model 2 plus body-mass index and personal history of diabetes. ‡P values for trend are based on the median apolipoprotein levels in quintiles of the controls.

FIG. 11. Association between apolipoprotein concentrations in VLDL and coronary heart disease in the complete study sample and by sex. * Model 1 was conditioned on matching factors. Model 2 was additionally adjusted for presence or absence of a parental history of coronary heart disease before the age of 60 years, alcohol intake and personal history of hypertension. Model 3 was adjusted for everything in model 2 plus body-mass index and personal history of diabetes. †P values for trend are based on the median apolipoprotein levels in quintiles of the controls. ‡Calculated as the P value for the interaction between sex and median apolipoprotein levels in quintiles of the controls.

FIG. 12. Association between baseline characteristics (complete study sample) and plasma levels of apoB in LDL. FIG. 12A is a table depicting the baseline covariates across quintiles of apoB in LDL with apoC-III. FIG. 12B is a table comprising the baseline covariates across quintiles of apoB in LDL without apoC-III. Data are means unless specified otherwise. * Quintiles were calculated using sex-specific apoB levels among controls, the reported medians correspond to a weighted average of values in men and women.

FIG. 13. Association between traditional lipid risk factors and coronary heart disease in the complete study sample. Model was conditioned on matching factors, and additionally adjusted for the presence or absence of a parental history of coronary heart disease before the age of 60 years, alcohol intake and personal history of hypertension. * P values for trend are based on the median levels of each variable in quintiles of the controls.

FIG. 14. Relative risk of coronary heart disease during follow up in the complete study sample, according to levels of apoB in LDL without apoC-III, in models including other major lipid risk factors. Each panel represents a separate model in which apoB and another lipid risk factor were mutually adjusted. Solid bars represent relative risks for quintile 5 compared to quintile 1 of each risk factor, and error bars represent 95% confidence intervals. All models were conditioned on matching factors, and additionally adjusted for the presence or absence of a parental history of coronary heart disease before the age of 60 years, alcohol intake and personal history of hypertension. * To be interpreted with caution, because the linear correlation coefficient between the two variables was 0.92.

FIG. 15. Relative risk of CHD during follow-up in the complete study sample, mutually adjusting levels of cholesterol in LDL with and without apoC-III. Relative risks and 95% confidence intervals are given for quintile 5 compared to quintile 1. The model was conditioned on matching factors, and additionally adjusted for the presence or absence of a parental history of coronary heart disease before the age of 60 years, alcohol intake and personal history of hypertension.

FIG. 16. Adjusted relative risks for CHD according to apoC-I and apoC-II in LDL.

FIG. 17. Relative risks for CHD according to apoC-II in LDL particles with and without apoC-III. ApoC-II in LDL with and without apoC-III simultaneously included in adjusted model, in addition to the concentration of apoC-III in LDL.

FIG. 18. Adjusted relative risk for CHD according to joint classification of tertiles of apoC-II in LDL and the proportion of LDL with apoC-III.

### DETAILED DESCRIPTION

### Introduction

Population studies have shown that low-density lipoprotein cholesterol (LDL-C) directly and high-density lipoprotein cholesterol (HDL-C) inversely predict risk of coronary heart disease (CHD) (Gordon et al., 1977, Am J Med 62:707-714; Sharrett et al., 2002, Circulation 104:1108-1113; Assmann et al., 1996, Atherosclerosis 124 (suppl):S11-S20; Di Angelantonio et al., 2009, JAMA 302:1993-2000; McQueen et al., 2008, Lancet 372:224-233). Although statins and other classes of drugs efficiently reduce LDL-C and concomitantly lower the risk of cardiovascular events (Grundy et al., 2004, J Am Coll Cardiol 44:720-732), evidence for independent atheroprotective effects of raising HDL-C is inconsistent (Singh et al., 2007, JAMA 298:786-798). The anti-atherogenic properties of the HDL particle include the ability to promote transport of cholesterol from peripheral tissues such as the artery wall to the liver, as well as anti-inflammatory, anti-apoptotic, nitric oxide-promoting, prostacyclin-stabilizing, and platelet-inhibiting functions (Assmann et al., 2004, Circulation 109:1118-11114). However, changes in HDL-C among some trials using hypolipidemic drugs did not independently predict changes in CHD (Singh et al., 2007, JAMA 298:786-798; Briel et al., 2009, BMJ 338:b92); and the lack of CHD reduction in trials of a drug that raises HDL-C by an unprecedented amount using a novel mechanism suggests the possibility that HDL-C may contain protective and nonprotective components (Nissen et al., 2007, N Engl J Med 356:1304-1316; Barter et al., 2007, N Engl J Med 357:2109-2122). In this regard, it has been demonstrated that the concentration in blood of apolipoprotein E in HDL independently and significantly predicted increased risk of CHD, and the concentration of apolipoprotein C-III in HDL trended in the same direction of increased risk (Sacks et al, Circulation 2000; 102:1886-1892).

The metabolic heterogeneity of HDL particles may underlie the inconsistency between epidemiological studies that consistently showed independent risk prediction and experimental approaches in clinical trials of lipid treatments that did not show that increased HDL concentrations correlated with decreased CHD rates. HDL comprises a diverse group of lipoproteins with substantial differences in size and density, and composition of lipids and proteins that influence the functional properties and metabolism of the particles. Thus, it is likely that subpopulations of HDL exist with more or less anti-atherogenic potential (Davidson et al., 2009, Arterioscler Thromb Vasc Biol 29:870-876; El Harchaoui et al., 2009, Ann Intern Med 150:84-93; Vaisar et al., 2007, J Clin Invest 117:746-756; Movva et al., 2008, Clin Chem 54:788-800). Several large-scale epidemiologic studies have investigated the risk of CHD when HDL was separated by size. In some population-based studies the concentration of small size HDL correlated with increased CHD but in other studies the concentration of small HDL predicted lower rates of CHD. In another type of study, an increase in small size HDL caused by the drug gemfibrozil was associated with lower incidence of CHD (Stampfer et al., 1991, N Engl J Med 325:373-381; Asztalos et al., 2008, Metabolism 57:77-83; Sweetnam et al., 1994, Circulation 90:769-774). In contrast in other studies, large size HDL had the protective associations (Asztalos et al., 2004, Arterioscler Thromb Vasc Biol 24:2181-2187; Mora et al., 2009, Circulation 119:931-939). Finally to the contrary, in another cohort study, very large HDL particle size was directly associated with incidence of CAD (van der Steeg et al., 2008, J Am Coll Cardiol 51:634-642). Thus, it remains inconclusive whether any of these techniques lead to any gain in information in terms of the identification of HDL subclasses with variable anti-atherogenic potential. Efforts to identify characteristics that may modulate the functional properties and metabolism of the HDL particle are important to improve the understanding of the atherosclerotic process and to prevent and treat cardiovascular diseases.

In previous work, it was found that apolipoprotein (apo)C-III, a small protein that resides on the surface of some lipoproteins (Gangabadage et al., 2008, J Biol Chem 283:17416-17427; Alaupovic, 1996, Methods Enzymol 263:32-60), provoked inflammatory and atherogenic responses in cells that are involved in atherosclerosis (Kawakami et al., 2006, Circulation 114:681-687; Kawakami et al., 2006, Circulation 113:691-700). The plasma concentration of apolipoprotein C-III (apoC-III) in VLDL and LDL, or the concentration of LDL that has apoC-III predicted risk of cardiovascular disease (CVD) or progression of coronary atherosclerosis independently of standard lipid risk factors (Sacks et al., 2000, Circulation 102:1886-1892; Lee et al., 2003, Arterioscler Thromb Vasc Biol 23:853-858; Alaupovic et al., 1997, Arterioscler Thromb Vasc Biol. 17:715-722; Mendivil et al., 2011, Circulation 124:2065-2072). Although HDL particles exist both with and without apoC-III, little is known about the role of apoC-III in relation to HDL function or risk of CHD. As mentioned, apoC-III in HDL showed a trend toward increased risk of CHD (Sacks et al., 2000, Circulation 102:1886-1892).

Apolipoprotein E (apoE) is a small apolipoprotein synthesized mostly by the liver (Mahley, 1988, Science 240:622-30; Mahley & Huang, 1999, Curr Opin Lipidol 10:207-17) that serves as a ligand to the LDL receptor (LDLR) and the LDL-receptor-related protein-1 (LRP1), and plays an essential role in metabolism by promoting uptake of lipoproteins by the liver. Even though most plasma apoE is borne in HDL and VLDL, there is a measurable concentration of apoE in LDL (Campos et al., 2001, J Lipid Res 42:1239-1249; Zheng et al., 2007, J Lipid Res 48:1190-1203). ApoE has a very high affinity for the LDL receptor, actually much superior to that of apoB-100 (Mahley & Innerarity, 1983, Biochim Biophys Acta 737:197-222), hence apoE in LDL may influence the plasma concentration and metabolic destination of LDL particles, with potential implications for atherogenesis and the occurrence of cardiovascular disease (CVD). In addition, apoE has diverse proposed antiatherogenic properties independent of its role on lipoprotein uptake (Curtiss, 2000, Arterioscler Thromb Vasc Biol 20:1582-53).

The aforementioned apolipoprotein C-III (apoC-III) has actions antagonistic to those of apoE. ApoC-III impedes binding of VLDL to receptors on liver cells, channeling the metabolism of VLDL away from clearance from the circulation and toward conversion to LDL, especially dense LDL (Zheng et al., 2007, J Lipid Res 48:1190-1203; Zheng et al., 2010, Circulation 121:1722-34; Mendivil et al., 2010, Arterioscler Thromb Vasc Biol 30:239-245). The plasma concentration of LDL with apoC-III predicts incidence of recurrent cardiovascular events in type 2 diabetes (Lee et al., 2003, Arterioscler Thromb Vasc Biol 23:853-858). Interestingly, most VLDL and LDL that contain apoE also contain apoC-III (Campos et al., 2001, J Lipid Res 42:1239-1249; Zheng et al., 2007, J Lipid Res 48:1190-1203). The amounts of apoE and apoC-III individually in VLDL and LDL vary substantially so the balance between the apoE and apoC-III content of an individuals' VLDL and LDL may greatly impact their physiology and subsequently the progression of atherosclerosis.

Even though genetic variations at the APOE gene have been extensively studied as predictors of cardiovascular events (Menzel et al., 1983, Arterioscler Thromb Vasc Biol 3:310-315; Davignon et al., 1988, Arterioscler Thromb Vasc Biol 8:1-21; Stengård et al., 1995, Circulation 91:265-9), very few studies have analyzed concentrations of the apoE protein in VLDL, LDL or LDL as predictors of cardiovascular outcomes, or how they are affected by concomitant concentrations of apoC-III.

One study in adults over 85 years (Mooijaart et al., 2006, PLoS Med 3(6):e176) found a positive association between total plasma apoE concentrations and cardiovascular mortality, and a second paper reported a generally increased risk of stroke with higher plasma apoE in the same cohort of older individuals (van Vliet et al., 2007, Ann N Y Acad Sci 1100:140-7). However, plasma total apoE is distributed among all lipoprotein classes, and a considerable amount of apoE is in HDL. As mentioned, the apoE content of HDL is associated with recurrent coronary heart disease events (Sacks et al., 2000, Circulation 102:1886-92). Thus, the presence of apoE may have a lipoprotein-specific influence on atherosclerosis and CHD. It is also not clear if the findings from this older cohort can be extrapolated to the general adult population.

Very low-density lipoprotein (VLDL) and low-density lipoprotein (LDL) are heterogeneous lipoprotein classes that vary in size and content of lipid and protein (Alaupovic, 1996, Methods Enzymol 263:32-60). Apolipoprotein (apo) B is the required structural apolipoprotein of VLDL and LDL. Each VLDL and LDL has only 1 molecule of apoB but may have no or many molecules of apoC-III attached to its surface (Alaupovic, 1996, Methods Enzymol 263:32-60; Campos et al., 2001, J Lipid Res 42:1239-1249; Lee et al., 2003, Arterioscler Thromb Vasc Biol 23:853-858). About 40% to 60% of VLDL and 10% to 20% of LDL have apoC-III (Campos et al., 2001, J Lipid Res 42:1239-1249; Lee et al., 2003, Arterioscler Thromb Vasc Biol 23:853-858). ApoC-III has deleterious effects on the metabolism of VLDL and LDL (Ooi et al., 2008, Clin Sci 114: 611-624; Mendivil et al., 2010, Arterioscler Thromb Vasc Biol 30:239-245; Zheng et al., 2007, J Lipid Res 48:1190-1203) and on functions of cells that participate in atherosclerosis (Kawakami A et al. Circulation 2006). ApoC-III interferes with the binding of VLDL to receptors in the liver, which inhibits the removal of VLDL from plasma (Zheng et al., 2007, J Lipid Res 48:1190-1203; Mendivil et al., 2010, Arterioscler Thromb Vasc Biol 30:239-245; Sehayek & Eisenberg, 1991, J Biol Chem 266:18259-18267). Nearly all VLDL that has apoC-III and not apoE undergoes intravascular lipolysis of its triglyceride content, producing LDL with apoC-III, and large LDL with apoC-III is metabolized to small LDL (Zheng et al., 2007, J Lipid Res 48:1190-1203; Mendivil et al., 2010, Arterioscler Thromb Vasc Biol 30:239-245; Zheng et al., 2010, Circulation 121:1722-1734).

During the last few years, evidence has accumulated indicating that the protein composition of lipoproteins may be more relevant to atherogenesis and cardiovascular risk than the absolute concentrations of plasma lipids. One of these compositional factors is apolipoprotein (apo)C-III, a small interchangeable apolipoprotein that impairs hepatic uptake of circulating lipoproteins and has various direct proatherogenic effects on the arterial wall.

ApoC-III-containing VLDL and LDL prepared from human plasma activate monocytes that circulate in blood to adhere to vascular endothelial cells, an early step in atherosclerosis (Kawakami et al., 2006, Circulation 114:681-687; Kawakami et al., 2006, Circulation 113:691-700). These actions are not shared by VLDL or LDL that does not have apoC-III. The presence of apoC-III on LDL is also associated with compositional changes that favor LDL adhesion to the subendothelial extracellular matrix (Hiukka et al., 2009, Diabetes 58: 2018-2026). Thus, apoC-III may trap its associated lipoproteins in the arterial wall and bring in blood monocytes, crucial steps in the initiation and progression of atherosclerosis. ApoC-III concentrations in VLDL and LDL are positively associated with the progression of atherosclerosis or risk of coronary heart disease (CHD) (Blankenhorn et al., 1990, Circulation 81:470-476; Sacks et al., 2000, Circulation 102:1886-1892; Luc et al., 1996, J Lipid Res 37:508-517; Hodis et al., 1994, Circulation 90: 42-49), and it had previously been reported that LDL with apoC-III is associated with recurrent cardiovascular disease among patients with a prior myocardial infarction and type 2 diabetes mellitus (Lee et al., 2003, Arterioscler Thromb Vasc Biol 23:853-858).

There is thus a need in the art for methods to assess risk of cardiovascular disease. The methods and compositions provided by some aspects of this disclosure address this unmet need.

Unless defined otherwise, all technical and scientific terms used herein have the same meaning as commonly understood by one of ordinary skill in the art. Where not otherwise indicated, gene and protein names used herein are official names according to the Human Genome Organization Gene Nomenclature Committee (see, e.g., Seal R L, Gordon S M, Lush M J, Wright M W, Bruford E A. genenames.org: the HGNC resources in 2011. Nucleic Acids Res. 2011 January; 39 (Database issue):D519-9. PMID: 20929869; and Shows, T B; McAlpine, P J; Boucheix, C; Collins, F S; Conneally, P M; Frézal, J; Gershowitz, H; Goodfellow, P N et al. (1987). Guidelines for human gene nomenclature. An international system for human gene nomenclature (ISGN, 1987). Cytogenetics and cell genetics 46 (1-4): 11-28. PMID 3507270; the entire contents of each of which are incorporated herein by reference).

The term “about,” when referring to a measurable value such as an amount, a temporal duration, and the like, is meant to encompass variations of no more than ±20%, no more than ±10%, no more than ±5%, no more than ±1%, or no more than ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “analyte” refers to any substance or chemical constituent that is undergoing analysis. For example, an “analyte” can refer to any atom and/or molecule; including their complexes and fragment ions. The term may refer to a single component or a set of components. In the case of biological molecules/macromolecules, such analytes include but are not limited to: polypeptides, polynucleotides, proteins, peptides, antibodies, DNA, RNA, carbohydrates, steroids, and lipids, and any detectable moiety thereof, e.g. immunologically detectable fragments. In the context of lipoproteins, an analyte can be a molecule comprised in a lipoprotein particle, for example, a protein or peptide (e.g., an integral or non-integral lipoprotein-associated protein or apolipoprotein), a lipid (e.g., a triacylglycerol, a cholesterol, or cholesterol derivative), or any other molecule or molecule type known to be comprised in or otherwise associated with lipoprotein particles. In some instances, an analyte can be a biomarker.

The term “apolipoprotein” refers to a protein that combines with lipids to form a lipoprotein particle. The lipoproteins that circulate in blood in humans are divided into two categories defined by their specific integral apolipoprotein-apo A-1 (or apoA-I) lipoproteins and apoB lipoproteins. See, e.g., Alaupovic P. Significance of apolipoproteins for structure, function, and classification of plasma lipoproteins. Methods Enzymol. 1996; 263:32-60. ApoA-I lipoproteins are commonly called high density lipoproteins (HDL), and apoB lipoproteins include chylomicrons, very low density lipoproteins, intermediate density lipoproteins, low density lipoproteins, and lipoprotein(a). The integral apolipoproteins are termed as such because they are essential for the synthesis of lipoproteins by the intestine and liver, and provide to the lipoproteins essential metabolic functions. ApoA-I is the integral apolipoprotein of HDL, and it is an activator of reverse cholesterol transport, the principal function of HDL that removes cholesterol from tissues including arteries containing atherosclerosis, packages the cholesterol in the HDL particle, and delivers it to the liver for excretion. A non-integral apolipoproteins is an apolipoproteins that is present on a lipoprotein but not required for its synthesis in the liver or intestine or its secretion into the blood circulation. The non-integral apolipoproteins regulate the metabolism of the lipoproteins once they are secreted into blood, for example by targeting the lipoproteins for uptake by specific cells (e.g. apoE) or by blocking lipoprotein clearance from plasma (e.g. apoC-III). Non-integral apolipoproteins also affect the risk of cardiovascular disease. The unique nature of the integral apolipoproteins is their stoichiometric relationship with lipoprotein particles, providing an estimate of the lipoprotein particle concentration. In contrast, the content of non-integral apolipoproteins can vary substantially from zero to more than 100 on a single lipoprotein particle.

The term “lipoprotein-associated protein,” as used herein is any protein that can be found in a lipoprotein particle. In some instances, the lipoprotein-associated protein is bound to a lipoprotein, and in other instances it is more loosely associated with a lipoprotein.

A “component” of an index refers to any measured analyte that is used in an algorithm for the determination of an index as provided herein.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

As used herein, the term “biomarker” is a biological compound such as a protein or a fragment thereof, including a polypeptide or peptide that may be isolated from, or measured in the biological sample, which is differentially present in a sample taken from a subject having established or potentially clinically significant CVD as compared to a comparable sample taken from an apparently normal subject that does not have CVD. A biomarker can be an intact molecule, or it can be a portion thereof that may be partially functional or recognized, for example, by a specific binding protein or other detection method. A biomarker is considered to be informative for CVD if a measurable aspect of the biomarker is associated with the presence of CVD in a subject in comparison to a predetermined value or a reference profile from a control population. Such a measurable aspect may include, for example, the presence, absence, amount, or concentration of the biomarker, or a portion thereof, in the biological sample, and/or its presence as a part of a profile of more than one biomarker. A measurable aspect of a biomarker is also referred to as a feature. A feature may be a ratio of two or more measurable aspects of biomarkers. A biomarker profile comprises at least one measurable feature, and may comprise two, three, four, five, 10, 20, 30 or more features. The biomarker profile may also comprise at least one measurable aspect of at least one feature relative to at least one external or internal standard.

As used herein, the term “cardiovascular disease” or “CVD,” generally refers to heart and blood vessel diseases, including atherosclerosis, coronary heart disease (CHD), cerebrovascular disease, and peripheral vascular disease. Cardiovascular disorders are acute manifestations of CVD and include myocardial infarction, stroke, angina pectoris, transient ischemic attacks, and congestive heart failure. Cardiovascular disease, including atherosclerosis, usually results from the build-up of cholesterol, inflammatory cells, extracellular matrix and plaque.

As used herein, the term “coronary heart disease” or “CHD” refers to atherosclerosis in the arteries of the heart causing a heart attack or other clinical manifestation such as unstable angina.

As used herein, the term “data” in relation to one or more analytes, or the term “analyte data” generally refers to data reflective of the absolute and/or relative abundance (level) of a product of an analyte in a sample. As used herein, the term “dataset” in relation to one or more analytes refers to a set of data representing levels of each of one or more analyte products of a panel of analytes in a reference population of subjects. A dataset can be used to generate a formula/classifier. According to one embodiment the dataset need not comprise data for each analyte product of the panel for each subject of the reference or clinical population.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. In some embodiments, the animal is a mammal. In some embodiments, the mammal is a human.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, analyte value transformations and normalizations (including, without limitation, those normalization schemes based on clinical parameters, such as gender, age, or ethnicity), rules and guidelines, statistical classification models, and neural networks trained on historical populations. Of particular use in combining CVD analytes and other analytes are linear and non-linear equations and statistical classification analyses to determine the relationship between levels of CVD analytes detected in a subject sample. In panel and combination construction, of particular interest are structural statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Support Vector Machines (SVM), Random Forest (RF), Partial Least Squares, Sparse Partial Least Squares, Flexible Discriminant Analysis, Recursive Partitioning Tree (RPART), as well as other related decision tree classification techniques, Nearest Shrunken Centroids (SC), stepwise model selection procedures, Kth-Nearest Neighbor, Boosting or Boosted Tree, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art.

An “increased risk of developing CVD” is used herein to refer to an increase in the likelihood or possibility of a subject developing CVD. This risk can be assessed relative to a subject's own risk, or with respect to a reference population, e.g., to an age-matched and/or gender-matched population, and/or to a population that does not have clinical evidence of CVD. The reference population may be representative of the subject with regard to approximate age, age group and/or gender.

An “increased risk of progressing CVD” is used herein to refer to an increase in the likelihood or possibility of a subject that already has CVD to have progressing CVD, that is to develop further serious complications like a heart attack or stroke. This risk can be assessed relative to a subject's own risk, or with respect to a reference population, e.g., to an age-matched and/or gender-matched population, and/or to that does not have clinical evidence of progressing CVD. The reference population may be representative of the subject with regard to approximate age, age group and/or gender.

The “level” of one or more analytes means the absolute or relative amount or concentration of the analyte in the sample.

The term “lipoprotein particle,” as used herein, refers to a spherical or discoidal particle that contains both protein and lipid.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vivo, ex vivo, in vitro, or in situ, amenable to the methods described herein. In certain non-limiting embodiments, the patient, subject, or individual is a mammal, a non-human mammal, a laboratory animal, a rodent, a rat, a mouse, a hamster, a cat, a dog, a farm animal, a cattle, a sheep, a goat, a pig, or a human.

As used herein, the term “predetermined value” refers to the amount of one or more analytes in biological samples obtained from the general population or from a select population of subjects. For example, the select population may be comprised of apparently healthy subjects, such as subjects who have not previously had any sign or symptoms indicating the presence of CVD. In another example, the predetermined value may be comprised of subjects having established CVD. The predetermined value can be a cut-off value, or a range. The predetermined value can be established based upon comparative measurements between apparently healthy subjects and subjects with established CVD, as described herein.

“Sample” or “biological sample” as used herein means a biological material isolated from a subject. The biological sample may contain any biological material suitable for detecting the desired analytes, and may comprise cellular and/or non-cellular material obtained from the subject.

A “therapeutic” treatment is a treatment administered to a subject who exhibits a sign or symptom of pathology, for the purpose of diminishing or eliminating that sign or symptom.

As used herein, “treating a disease or disorder” means reducing the frequency with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder associated with CVD, including alleviating signs and symptoms of such diseases or disorders.

### An Index for Cardiovascular Disease Risk

Some aspects of this disclosure are based, at least in part, on the novel discovery that the detection and/or quantification of proteins that associate with a lipoprotein (referred to herein as a “lipoprotein-associated protein”) is useful for the generation of an index as a measure of a subject's risk of having or developing cardiovascular disease or of having a cardiovascular event.

An index as provided herein is thus useful, for example, as a diagnostic tool to assess a subject's cardiovascular disease risk, to develop a course of treatment for the subject, to assess the efficacy of drugs designed to treat cardiovascular disease, and/or to assess individual treatment protocols, on-going therapy in a subject, and the like. In some embodiments, an index provided herein is also useful as a research tool, e.g., for identifying compounds that have a desired effect on cardiovascular disease risk as assessed by monitoring changes in index scores and, thus, disease risk, effected by administering a candidate compound to a subject.

Some aspects of this disclosure provide methods for generating an index for assessing the risk of a subject for having or developing cardiovascular disease. In some embodiments, the index comprises risk factors for developing cardiovascular disease, e.g., as described herein. In some embodiments, the index comprises cardiovascular disease-protective factors, e.g., as described herein. In some embodiments, the index comprises both risk factors and protective factors. In some embodiments, an index score is calculated based on measurements of the respective risk or protective factors in the subject. In some embodiments where both risk factors and protective factors are assessed and used as a basis for the calculation of an index score, risk factors are scored inversely from protective factors, e.g., in that the detection of the presence or a high level of a risk factor in the subject leads to an increase in the index score, while detection of the presence or a high level of a protective factor in the subject leads to a decrease in the index score, or vice versa (risk factor decreases index score while protective factor increases it. In some embodiments, the respective index is generated by detecting a risk factor or protective factor in the subject by detecting an associated analyte, e.g., a component of a lipoprotein, as disclosed herein, in a biological sample derived from the subject, and by generating the index based on the detection (or lack thereof) of the risk and/or protective factor(s).

Some exemplary suitable analytes associated with risk for or protection from having or developing cardiovascular disease are provided herein, e.g., in Table 1. Additional suitable analytes will be apparent to those of skill in the art based on the instant disclosure. In some embodiments, detecting the respective analyte(s) comprises measuring the presence or a level or concentration of the analyte, e.g., via a quantitative or semi-quantitative assay. Some exemplary suitable assays for analyte measurements are provided herein, and additional assays will be apparent to those of skill in the art. In some embodiments, generating the index comprises calculating an index score based on the detection and/or quantification of the analyte. In some embodiments, an index generated according to some aspects of this invention is a VLDL-LDL Atherogenicity Index, an HDL Protection Index, a Global Lipoprotein Index, or any combination of these indices.

Some aspects of this disclosure provide a method comprising detecting the level of one or more lipoprotein-associated protein(s) in a lipoprotein and generating an index therefrom that is a measure of the subject's risk of having CVD. In some instances, the lipoprotein-associated protein can be an apolipoprotein. In some embodiments, the lipoprotein-associated protein includes but is not limited to those listed in Table 1. In some embodiments, the characterization of the content of lipoproteins with respect to the absence, presence, quantity or proportion of one or more of the lipoprotein-associated proteins provides an index for assessing the risk of CVD in a subject.

In some embodiments, the index for assessing risk of CVD in a subject includes detecting the level of at least one integral and at least one nonintegral lipoprotein-associated protein in a lipoprotein. In some embodiments, the index includes detecting the level of an integral and at least two nonintegral lipoprotein-associated proteins in a lipoprotein.

Some aspects of this disclosure provide a VLDL-LDL Atherogenicity Index and an HDL Protection Index. In some embodiments, each index takes into account levels of lipoproteins, such as measured by their content of their unique integral lipoprotein, apoB in VLDL and LDL and apoA-I in HDL; and at least one of the corresponding nonintegral lipoprotein-associated proteins, each of which is considered a component of the index. In some embodiments, each component is given a ranking according to the population distribution, wherein a component that has a strong relation to cardiovascular disease is designated to have a higher possible score than a component that has a weaker relation to cardiovascular disease. For example, a ranking of zero may be given to a component that is in the first quintile, i.e. below the 20^{th} percentile; a ranking of 1 may be given for the second quintile; a ranking of 2 for the 3^{rd} quintile, a ranking of 3 for the 4^{th} quintile and a ranking of 4 for the 5^{th} quintile, i.e. above the 80^{th} percentile. To the contrary, the ranking for a protective component to cardiovascular disease is typically opposite to that of a component having a strong relation to cardiovascular disease. For example, a ranking of zero may be assigned for the 5^{th} quintile, a ranking of 1 may be assigned for the 4^{th} quintile, a ranking of 2 may be assigned for the 3^{rd} quintile, a ranking of 3 may be assigned for the 2^{nd} quintile, and a ranking of 4 may be assigned for the 1^{st} quintile. In this way, each index determines to what extent the combination of components contribute significantly to the level of risk an individual has of having a cardiovascular disease. The indices described herein can be used alone, or can be combined into a single index, that is descriptive of, and predictive of, the overall impact on cardiovascular disease. A more detailed description of some exemplary indices provided herein is discussed elsewhere herein.

In some embodiments, the VLDL-LDL Atherogenicity Index comprises a measure of apoC-II, apoC-III, apoE levels, or combinations thereof. However, this disclosure is not limited to measuring only apoC-II, apoC-III, and apoE levels in the context of VLDL-LDL. Rather, any lipoprotein-associated protein disclosed herein, or to be identified in the future, can be used to generate a VLDL-LDL Atherogenicity Index. In certain embodiments, a high index number for the VLDL-LDL Atherogenicity Index is defined by a high amount of apoC-II and apoC-III, in combination with a low amount of apoE, on VLDL and LDL. In such embodiments, thus, a subject who has a high VLDL-LDL Atherogenicity Index value would have a high risk of having a cardiovascular disease, whereas a subject who has a low VLDL-LDL Atherogenicity Index number would have a low risk of having a cardiovascular disease.

In some embodiments, the HDL Protection Index comprises a measure of apoC-III, apoE levels, or combinations thereof. In some embodiments, the HDL Protection Index comprises a measure of apoE concentration (or apoE/apoA-I) of HDL and/or HDL with or without apoC-III. However, this disclosure is not limited to measuring only apoC-III and apoE levels in the context of HDL. Rather, any lipoprotein-associated protein disclosed herein, or to be identified in the future, can be used to generate an HDL Protection Index. In certain embodiments, a subject who has a high HDL Protection Index number would have a low level of HDL with apoC-III and apoE, while a subject who has a low HDL Protection Index number would have a high level of HDL with apoC-III and apoE. In such embodiments, thus, a subject who has a high HDL Protection Index value would have a low risk of having a cardiovascular disease, whereas a subject who has a low HDL Protection Index number would have a high risk of having a cardiovascular disease.

In some embodiments, both the VLDL-LDL Atherogenicity and the HDL Protection Index of a subject are determined and are considered in combination with each other to generate a Global Lipoprotein Index to aid in the prediction of the overall risk that a subject has, or will develop a cardiovascular disease. The Global Lipoprotein Index may be constructed, for example, as a simple sum of the two individual indexes; as produced by multiplying the indexes; as produced by mathematical modeling; or by a simple ratio of the VLDL-LDL Atherogenicity Index and the HDL Protection Index.

Some aspects of this disclosure provide a database of one or more of VLDL, LDL, HDL, apolipoproteins, and other lipoprotein-associated proteins listed in Table 1, together with patient data useful for treatment, diagnosis, and monitoring cardiovascular disease. The database contains, in some embodiments, quantitative lipoprotein and lipoprotein-associated protein (e.g., apolipoprotein) data and permits deriving relationships amongst the lipoprotein and lipoprotein-associated protein values and cardiovascular disease. In some embodiments, the data can be summarized as a VLDL-LDL Atherogenicity Index and an HDL Protection Index. Quantitative data typically permits more effective treatment and monitoring of cardiovascular disease. In an example, the VLDL-LDL Atherogenicity Index can be used as a target for treatment, for example by diet or drugs, and a treatment-related reduction can be used as an indicator of success of the treatment. In a further example, the HDL Protection Index can be used as a target for treatment by diet or drugs in which an increase is indicative of success of the treatment. Quantitative differences in the VLDL-LDL Atherogenicity Index and the HDL Protection Index together can optimize the need for and success of more or less aggressive treatment. Thus, in some embodiments, the combination of the VLDL-LDL Atherogenicity Index and HDL Protection Index provides a Global Lipoprotein Index that can be used to more effectively treat and monitor cardiovascular disease.

Some aspects of this disclosure provide compositions for use in the methods described herein. In some embodiments, the composition comprises a reagent that detects and/or quantitates an analyte. In some embodiments, the composition comprises a panel of reagents, each of which detects and/or quantitates a different analyte. Suitable analytes include, but are not limited to, one or more of vLDL, LDL, HDL, apolipoprotein, and a lipoprotein-associated protein listed in Table 1.

Some aspects of this disclosure relate to the characterization of lipoproteins by their association with a lipoprotein-associated protein for the determination of cardiovascular disease risk. Some aspects of this disclosure provide methods for the diagnosis, early detection, risk estimation and monitoring of the course of disease in its untreated or treated state, in which the presence or amount of one or more lipoprotein-associated proteins is determined in association with a lipoprotein type such as HDL. Non-limiting examples of a lipoprotein-associated protein includes but is not limited to the proteins listed in Table 1. Accordingly, the characterization of lipoproteins that uses the lipoprotein-associated protein content provides an index for assessing the risk of a disease, for example, a cardiovascular disease.

TABLE 1
exemplary lipoprotein-associated proteins
Protein Name

a-1-acid glycoprotein 1
a-1-acid glycoprotein 2
a-1-antichymotrypsin
a-1-antitrypsin
a-1B-glycoprotein
a-1-microglobulin/bikunin
a-2-antiplasmin
a-2-HS-glycoprotein
Afamin
aminopeptidase N
Angiotensinogen
antithrombin III
apo (a)
apoA-I
apoA-II
apoA-IV
ApoB
apoB-100
apoC-I
apoC-II
apoC-III
apoC-IV
ApoD
ApoE
ApoF
ApoH
apoL-I
ApoM
b-2-microglobulin
band 3 anion transport protein
carbonic anhydrase 1
catheliciden antimicrobial
peptide
CETP
Clusterin (apoJ)
complement B
complement C1s subcomponent
Complement C3
Complement C4A
Complement C4B
Complement C9
Fibrinogen (alpha-chain)
Fibronectin
filamin-A
Gelsolin
Haptoglobin-related protein
hemoglobin binding protein
hemoglobin subunit alpha
hemoglobin subunit beta
Hemopexin
heparin cofactor 2
integrin a-II-b
Inter-a-trypsin inhibitor H2
Inter-a-trypsin inhibitor H4
Kininogen-1
LCAT (phosphatidylcholine-
sterol acyltransferase)
leucine-rich a-2 glycoprotein
Lipoprotein Associated
Phospholipase A2 (PAF-AH)
Lumican
N-acetylmuramoyl-L-alanine
amidase
phosphatidylinositol-glycan-
specific phospholipase D
pigment epithelium derived
factor
PL transfer protein
Plasma retinol-binding protein
platelet basic protein
PON 1
PON 3
Prenylcysteine oxidase
Prothrombin
SAA1
SAA2
SAA4
Serotransferrin
Serpin peptidase inhibitor
Serum albumin
thrombospondin I
Transthyretin
Vitamin D-binding protein
Vitronectin
zinc a-2 glycoprotein

In certain embodiments, methods as provided herein can be used for permitting refinement of disease diagnosis, disease risk determination, and clinical management of subjects having, or at risk of developing cardiovascular disease. In some embodiments, an index provides herein represents a matrix for assessing risk of having or developing cardiovascular disease. In some embodiments, the detection of the selective analytes to generate an index as provided herein in subjects, or samples obtained therefrom, permits refinement of disease diagnosis, disease risk determination, and clinical management of subjects being treated with agents that are associated with cardiovascular disease.

### Lipoprotein and Lipoprotein-Associated Protein

Some aspects of this disclosure relate to the detection of lipoprotein-associated protein (e.g, apolipoprotein and proteins listed in Table I) content associated with a specific lipoprotein density class to generate an index that is useful for a variety of applications described elsewhere herein.

Types of lipoproteins include high density lipoprotein (HDL), low density lipoprotein (LDL), intermediate density lipoprotein (IDL), very low density lipoprotein particles (VLDL), chylomicron (CM), and lipoprotein(a) (Lp(a)). Partially hydrolyzed VLDL is called intermediate density lipoproteins (IDL) that themselves can be hydrolyzed further to LDL. Each varies in size, density, protein composition, and lipid composition.

Proteins which form lipoproteins together with lipids (triglycerides, cholesterol, phospholipids) are designated herein as “lipoprotein-associated protein” wherein some lipoprotein-associated protein is considered to be “apolipoproteins.” An important function of the apolipoproteins is to permit the transport of the water-insoluble lipids in serum and plasma. On the basis of their migration behavior in the density gradient of an ultracentrifuge, lipoproteins are assigned to different density classes: VLDL (very low-density lipoproteins), LDL (low-density lipoproteins) and HDL (high-density lipoproteins). Lipoproteins of different density classes differ not only on the basis of the amount and type of lipids present altogether but also with regard to the composition thereof. It is furthermore possible to assign typical apolipoprotein profiles to the respective density class.

Apolipoproteins are proteins of different length and amino acid composition. The primary structures (amino acid sequences) of the various apolipoproteins are known, and data or specific concepts regarding their three-dimensional structure also exist for a number of different apolipoproteins, in particular in association with lipids.

Classes and subclasses of apolipoproteins are apolipoprotein A (Apo A-I, Apo A-II, Apo A-IV, and Apo A-V), apolipoprotein B (Apo B-48 and Apo B-100), apolipoprotein C (Apo C-I, Apo C-II, Apo C-III, and Apo C-IV), apolipoprotein D, apolipoprotein E (Apo E-2, E-3, and E-4), apolipoprotein H (Apo H), and apolipoprotein J (Apo J), among others.

Different lipoprotein particles have different apolipoproteins on their surface. Apolipoproteins present in HDL are Apo A-I, A-II, A-IV, A-V, C-I, C-II, D, E-2, E-3, and E-4, and Apo J. In some instances, lipoprotein-associated proteins present in HDL can include one or more proteins listed in Table 1.

The integral apolipoprotein in LDL is Apo B-100. LDL also can contain apoC-I, C-II, C-III, E, and the like. In some instances, lipoprotein-associated proteins present in LDL can include one or more proteins listed in Table I.

Apolipoproteins in IDL are Apo B-100, C, E-2, E-3, and E-4. In some instances, lipoprotein-associated proteins present in IDL can include one or more proteins listed in Table I.

Apolipoproteins in VLDL are Apo A-V, B-100, C-I, C-II, C-IV, E-2, E-3, and E-4. In some instances, lipoprotein-associated proteins present in VLDL can include one or more proteins listed in Table 1.

Apolipoproteins in chylomicrons are Apo A-I, A-II, A-IV, B-48, C-I, C-II, C-III, and E-2, E-3, and E-4. In some instances, lipoprotein-associated proteins present in chylomicrons can include one or more proteins listed in Table I.

Some aspects of this disclosure provide methods related to the determination of levels of a lipoprotein-associated protein (e.g., an apolipoprotein), including both integral and non-integral proteins that are associated with LDL, HDL, etc., which levels are used to generate an index. Some aspects of this disclosure relate to the detection or quantification of a desired lipoprotein-associated protein, e.g., an apolipoprotein, as well as “derivatives thereof” which includes fragments and aggregates, in particular those which behave like the free apolipoprotein in the respective chosen assay method. The “derivatives” may be, for example, an apolipoprotein molecule shortened by individual amino acids or amino acid sequences, or complete apolipoprotein molecule sterically or conformationally modified, for example by aggregation.

Some of the novel discoveries which permit the use of a desired lipoprotein-associated protein (e.g., apolipoprotein) as an analyte indicate that, in patients who are suffering from relevant diseases, the detectable amount of a desired apolipoprotein is not at an optimal level compared with apparently normal healthy persons.

Some embodiments provide a method of measuring the amount of apoC-III in VLDL and LDL in order to generate an index, wherein higher levels of apoC-III in VLDL and LDL is a predictor of cardiovascular disease.

Some embodiments provide a method of measuring HDL with apoC-III. For example, high HDL levels are generally believed to be associated with low risk of cardiovascular disease. However, when apoC-III is associated with HDL, the subject is generally believed to be at high risk for cardiovascular disease. However, when apoC-III is not associated with HDL, the subject is generally believed to be at a lower risk for cardiovascular disease, which is the usual association that HDL has. Therefore, a large amount of HDL that has apoC-III associated therewith, is presumably a dysfunctional form of HDL that does not have cardioprotective benefits, while a low amount of HDL that has apoC-III or a high amount of HDL without apoC-III is an indicator of protection against cardiovascular disease.

Other non-integral apolipoproteins are also known to affect the metabolism, atherogenicity, or risk associated with LDLs and HDLs. For example, it was found that the presence of apoE on VLDL and LDL that have apoC-III mitigated the high risk for cardiovascular disease of these types of VLDL and LDL. With respect to apoC-II, the presence of apoC-II on VLDL and LDL with apoC-III is associated with an increased high risk of cardiovascular disease to the already high risk of cardiovascular disease associated with these types of lipoproteins.

With respect to apoC-I, the presence of apoC-I in VLDL and LDL with apoC-III is associated with an increased high risk of cardiovascular disease.

Accordingly, some aspects of this disclosure provide a VLDL-LDL Atherogenicity Index where an increased amount of apoC-II and apoC-III and decreased amount of apoE on VLDL and LDL is a diagnosis of higher risk of cardiovascular disease.

With respect to HDL, the amount of apoE on HDL was observed to be associated with high risk of cardiovascular events. Accordingly, some aspects of this disclosure provide an HDL Protection Index where for example, an increase amount of the combination of apoC-III and apoE on HDL corresponds to an increased risk of cardiovascular disease.

Some aspects of this disclosure provide sensitive and rapid methods that can be useful in identifying and assessing cardiovascular diseases (e.g. lipid disorders, metabolic syndrome, and atherosclerosis), and any condition or disorder that may be diagnosed and characterized using a profile comprising lipoprotein and lipoprotein-associated protein (e.g., apolipoprotein) content. The profile comprising lipoprotein and lipoprotein-associated protein (e.g., apolipoprotein) content allows for the generation of indices that can be used to sensitively and rapidly assess cardiovascular diseases or categorizing one or more diseases or conditions. The profile data can be summarized as a VLDL-LDL Atherogenicity Index and an HDL Protection Index, wherein the combination of both indices provides a Global Lipoprotein Index.

In some embodiments, the indices provided herein are generated by calculating the amount of one or more of VLDL, LDL, HDL, apolipoprotein, and a lipoprotein-associated protein listed in Table 1.

VLDL-LDL Atherogenic Index

In some embodiments, the VLDL-LDL Atherogenic Index is calculated using an algorithm discussed elsewhere herein wherein an increase in the VLDL-LDL Atherogenic Index is indicative of an increase risk in cardiovascular disease. For example, in some embodiments, each component of the index is quantified in a sample of a subject. In some embodiments, each component in a sample of a subject is given a ranking according to the population distribution. In some embodiments, the rankings of the components are summed to produce the index. For example, apoC-III in VLDL+LDL is ranked according to quintiles of the population. In some embodiments, the ranking is specific for males and females. In some embodiments, a ranking of zero is given for apoC-III in VLDL+LDL levels that are in the first quintile, i.e. below the 20^{th} percentile; a ranking of 1 is given for the second quintile; a ranking of 2 for the third quintile, a ranking of 3 for the 4^{th} quintile and a ranking of 4 for the 5^{th} quintile, i.e. above the 80^{th} percentile.

As a non-limiting example, the ranking for apoC-II in VLDL+LDL is assigned in the same way as apoC-III in VLDL+LDL. However, the ranking for apoE in VLDL+LDL is opposite to that of apoC-II and apoC-III. A ranking of zero is assigned for the 5^{th} quintile, a ranking of 1 is assigned for the 4^{th} quintile, a ranking of 2 is assigned for the 3^{rd} quintile, a ranking of 3 is assigned for the 2^{nd} quintile, and a ranking of 4 is assigned for the 1^{st} quintile. The rankings are summed to yield the VLDL+LDL atherogenicity index. For example, a subject who has VLDL+LDL apoC-III in the 5^{th} quintile, apoC-II in the 5^{th} quintile, and apoE in the 1^{st} quintile is given the highest score, 12, indicating highest risk.

These scores for the components of the index are provided as an illustrative example and not meant to be limiting. The scores may be indexed to actual relative risks of each component as demonstrated by epidemiological studies such as those discussed elsewhere herein. In this way, a component that has a strong relation to cardiovascular disease will have a higher possible score than a component that has a weaker relation to cardiovascular disease. The summed scores, whatever the method of assignment are convertible to actual risks, relative and absolute, of the subject acquiring cardiovascular disease.

In other exemplary methods, protective components, such as VLDL+LDL apoE, are ranked on a negative scale, and in the summation, their influence is subtracted from the harmful components. For example, VLDL+LDL apoE in the 1^{st} quintile is given a score of zero, in the 2^{nd} quintile a score of minus 1, in the 3^{rd} quintile a score of minus 2, in the 4^{th} quintile a score of minus 3, in the 5^{th} quintile a score of minus 4. In the previous example, the subject has a score of 8 (apoC-III=4, apoC-II=4, apoE=zero). If that subject has an apoE level in the 5^{th} quintile, its score of minus 4 is subtracted from the scores of apoC-III and apoC-II to yield an index score of 4. In this method, the index can be converted into risk of cardiovascular disease, relative and absolute.

In another example, multiplicative scales are used such that the actual relative risks of the level of each component is multiplied rather than added. In this method, a protective component like apoE has a relative risk that is less than 1, i.e. zero to 0.99.

In some embodiments, each component is computed as a ratio of the non-integral apolipoprotein to the integral apolipoprotein, e.g. apoC-III divided by apoB. The ratios are given scores according to the quintile rankings or actual relative risks and then summed or multiplied to yield the index. The components of the indices can be expressed as concentrations of the non-integral apolipoproteins, or concentrations of the integral apolipoprotein (e.g., Apo A) that are associated with the non-integral apolipoprotein, e.g. the concentration of apoB that is associated with apoC-III (VLDL+LDL with apoC-III).

HDL Protection Index

In some embodiments, the HDL Protection Index is calculated using an algorithm discussed elsewhere herein wherein an increase in the HDL Protection Index is indicative of a decreased risk in cardiovascular disease.

In some embodiments, the components of the HDL Protection Index are given a score based on the quintile ranking. For example, apoC-III in HDL is ranked according to quintiles of the population. In some embodiments, the ranking is specific for males and females. In some embodiments, a ranking of 4 is given for apoC-III in HDL levels that are in the first quintile, i.e. below the 20^{th} percentile; a ranking of 3 is given for the second quintile; a ranking of 2 for the third quintile, a ranking of 1 for the 4^{th} quintile and a ranking of 0 for the 5^{th} quintile, i.e. above the 80^{th} percentile. Then, in some embodiments, apoE in HDL is ranked according to quintiles of the population. In some embodiments, the ranking is specific for males and females. In some embodiments, a ranking of 4 is given for apoE in HDL levels that are in the first quintile, i.e. below the 20^{th} percentile; a ranking of 3 is given for the second quintile; a ranking of 2 for the third quintile, a ranking of 1 for the 4^{th} quintile and a ranking of 0 for the 5^{th} quintile, i.e. above the 80^{th} percentile.

In some embodiments, the ranking for HDL without apoC-III or apoE is opposite to that of HDL apoC-III or HDL apoE. For example, in some embodiments, a ranking of 4 is assigned for the 5^{th} quintile, a ranking of 3 is assigned for the 4^{th} quintile, a ranking of 2 is assigned for the 3^{rd} quintile, a ranking of 1 is assigned for the 2^{nd} quintile, and a ranking of 0 is assigned for the 1^{st} quintile. In some embodiments, the rankings are summed to yield the HDL Protection Index. For example, a subject who has HDL apoC-III in the 5^{th} quintile, apoE in the 5^{th} quintile, and HDL without apoC-III or apoE in the 1^{st} quintile is given the lowest score, zero, indicating least protection by HDL and thus highest risk.

These scores for the components of the index are meant to be illustrative of some exemplary embodiments, and not meant to be limiting. The scores may be indexed to actual relative risks of each component as demonstrated by epidemiological studies such as those summarized later in this document and appended. In this way, a component that has a strong relation to cardiovascular disease will have a higher possible score than a component that has a weaker relation to cardiovascular disease. The summed scores, whatever the method of assignment are convertible to actual risks, relative and absolute, of the subject acquiring cardiovascular disease.

In some methods provided herein, protective components, such as HDL without apoC-III or apoE, are ranked on a negative scale, and in the summation, their influence is subtracted from the harmful components. For example, in some embodiments, HDL without apoC-III or apoE in the 1^{st} quintile is given a score of zero, in the 2^{nd} quintile a score of minus 1, in the 3^{rd} quintile a score of minus 2, in the 4^{th} quintile a score of minus 3, in the 5^{th} quintile a score of minus 4. Recomputing the previous example, the subject has a score of zero (HDL with apoC-III=0, HDL with apoE=0, HDL without apoC-III or apoE=zero). If that subject has an HDL without apoC-III or apoE level in the 5^{th} quintile, its score of minus 4 is subtracted from the scores of HDL with apoC-III and apoC-II to yield an index score of 4. Again, in this exemplary method, the index can be converted into risk of cardiovascular disease, relative and absolute. In another example, multiplicative scales are used such that the actual relative risks of the level of each component is multiplied rather than added. In this method, a protective component like HDL without apoC-III or apoE has a relative risk that is less than 1, i.e. zero to 0.99. In some embodiments, each component is computed as a ratio of the non-integral apolipoprotein to the integral apolipoprotein, e.g. apoC-III divided by apoA-I. The ratios are given scores according to the quintile rankings or actual relative risks and then summed or multiplied to yield the index.

In some methods provided herein, harmful components, such as HDL with apoC-III or apoE, are ranked on a negative scale, and in the summation, their influence is subtracted from the protective components. For example, HDL with apoC-III or apoE in the 1^{st} quintile is given a score of zero, in the 2^{nd} quintile a score of minus 1, in the 3^{rd} quintile a score of minus 2, in the 4^{th} quintile a score of minus 3, in the 5^{th} quintile a score of minus 4. Recomputing the previous example (HDL apoC-III in the 5^{th} quintile, apoE in the 5^{th} quintile, and HDL without apoC-III or apoE in the 1^{st} quintile), the subject has a score of minus 12 (HDL with apoC-III=−4, HDL with apoE=−4, HDL without apoC-III or apoE=−4). Again, in this method, the index can be converted into risk of cardiovascular disease, relative and absolute. In another example, multiplicative scales are used such that the actual relative risks of the level of each component is multiplied rather than added. In this exemplary method, a harmful component like HDL with apoC-III or apoE has a relative risk that is less than 1, i.e. zero to 0.99. In some embodiments, each component is computed as a ratio of the non-integral apolipoprotein to the integral apolipoprotein, e.g. apoC-III divided by apoA-I. In some embodiments, the ratios are given scores according to the quintile rankings or actual relative risks and then summed or multiplied to yield the index.

In some embodiments, the HDL Protection Index can be constructed from two or more separate HDL Indices: (1) Classical apolipoprotein index, e.g. apoC-III, apoE, apoA-II, etc.; (2) Thrombogenic index, e.g. prothrombin, anti-thrombin III, complement; (3) Inflammation index, e.g. SAA, etc.; (4) anti-oxidant index, e.g. PON, etc. The components of the indices can be expressed with as concentrations of the non-integral apolipoproteins, or concentrations of apoA-I that are associated with the non-integral apolipoprotein, e.g. the concentration of apoA-I that is associated with apoC-III (HDL with apoC-III).

In other embodiments, the VLDL+LDL and the HDL components may be computed as the cholesterol or triglyceride concentrations. For example, HDL apoC-III may be quantified as the cholesterol concentration of HDL that has apoC-III; and similarly for HDL apoE, and other components.

In some instances, the Atherogenicity and Protective Indices are summed or multiplied, as appropriate to the specific embodiment described elsewhere herein, to yield an overall cardiovascular disease risk index.

### Methods

Some aspects of this disclosure provide methods for qualifying risk of disease in a subject comprising generating a VLDL-LDL Atherogenicity Index and/or an HDL Protection Index. In some embodiments, the method comprises detecting an amount of a lipoprotein-associated protein, such as one or more from those listed in Table 1, found in a lipoprotein from a biological sample of the subject. In some embodiments, regardless of the index, a lipoprotein-associated protein that has a strong relation to cardiovascular disease is designated to have a higher possible score than a lipoprotein-associated protein that has a weaker relation to cardiovascular disease. Therefore, a higher VLDL-LDL Atherogenicity Index number is indicative of an increased risk of cardiovascular disease in such embodiments. In contrast, in some embodiments, a higher HDL Protection Index number is indicative of a decreased risk of cardiovascular disease. In some embodiments, the combination of the VLDL-LDL Atherogenicity Index and an HDL Protection Index provides a Global Lipoprotein Index that can be used to more effectively detect, diagnosis, treat and monitor cardiovascular disease.

In some embodiments, a VLDL-LDL Atherogenicity Index is generated by (1) measuring at least one analyte in a sample from the subject, wherein the at least one analyte is a lipoprotein-associated protein that is associated with VLDL-LDL, (2) analyzing or quantifying or measuring the at least one analyte in the sample, (3) preparing a profile of the at least one analyte using the analysis, quantification, or measurement; and (4) comparing the profile of the at least one analyte to standard profiles that indicate disease, whereby the presence, absence, or relative concentration of the at least one analyte in the sample indicates disease. In some embodiments, the lipoprotein-associated protein that is associated with VLDL-LDL is selected from the group consisting of any one of the proteins listed in Table 1, apoA-II, apoA-IV, apoA-V, apoC-I, apoC-II, apoC-III, apoC-IV, apoD, apoE, apoH, apoJ, apo-L, and any combinations thereof. In some embodiments, the amount of lipoprotein-associated protein that has a strong relation to cardiovascular disease is designated to have a higher possible score than a lipoprotein-associated protein that has a weaker relation to cardiovascular disease. In some embodiments, each lipoprotein-associated protein is ranked according to the population distribution. In some embodiments, the rankings of the components are summed to produce the index as discussed elsewhere herein.

Some aspects of this disclosure provide methods for assessing the risk of a patient having or developing cardiovascular disease. In some embodiments, the method comprises isolating in a biological sample obtained from the patient one or more of a VLDL and a LDL fraction, or a subfraction thereof; and measuring in the VLDL and/or LDL fraction, or subfraction thereof, the concentration of one or more lipoprotein-associated protein disclosed herein. In some embodiments, the one or more lipoprotein-associated protein is selected from the group consisting of any one of the proteins listed in Table 1, apoA-II, apoA-IV, apoA-V, apoC-I, apoC-II, apoC-III, apoC-IV, apoD, apoE, apoH, apoJ, apo-L, and any combinations thereof.

In some embodiments, an HDL Protection Index is generated by (1) measuring at least one analyte in a sample from the subject, wherein the at least one analyte is a lipoprotein-associated protein that is associated with HDL, (2) analyzing or quantifying or measuring the at least one analyte in the sample, (3) preparing a profile of the at least one analyte using the analysis, quantification, or measurement; and (4) comparing the profile of the at least one analyte to standard profiles that indicate disease, whereby the presence, absence, or relative concentration of the at least one analyte in the sample indicates disease. In some embodiments, the lipoprotein-associated protein that is associated with HDL is selected from the group consisting of any one of the proteins listed in Table 1, apoA-II, apoA-IV, apoA-V, apoC-I, apoC-II, apoC-III, apoC-IV, apoD, apoE, apoH, apoJ, apo-L, and any combinations thereof. In some embodiments, the amount of lipoprotein-associated protein that has a strong protective relation to cardiovascular disease is designated to have a higher possible score than a lipoprotein-associated protein that has a weaker protective relation to cardiovascular disease. In some embodiments, each lipoprotein-associated protein is ranked according to the population distribution. In some embodiments, the rankings of the components are summed to produce the index as discussed elsewhere herein.

Some aspects of this disclosure provide methods for assessing the risk of a patient having or developing cardiovascular disease. In some embodiments, the method comprises isolating in a biological sample obtained from the patient one or more of a HDL fraction, or a subfraction thereof; and measuring in the HDL fraction or subfraction thereof the concentration of one or more lipoprotein-associated protein disclosed herein. In some embodiments, the one or more lipoprotein-associated protein is selected from the group consisting of any one of the proteins listed in Table 1, apoA-II, apoA-IV, apoA-V, apoC-I, apoC-II, apoC-III, apoC-IV, apoD, apoE, apoH, apoJ, apo-L, and any combinations thereof.

The level of an analyte may be assessed in several different biological samples, for example bodily fluids. Non-limiting examples of bodily fluid include whole blood, plasma, serum, bile, lymph, pleural fluid, semen, saliva, sweat, urine, and CSF. In some embodiments, the bodily fluid is selected from the group of plasma and serum. In some embodiments, the bodily fluid is plasma. In some embodiments, the bodily fluid is serum.

In some embodiments, the bodily fluid is obtained from the subject using conventional methods in the art. For instance, one skilled in the art knows how to draw blood and how to process it in order to obtain serum and/or plasma for use according to aspects of this disclosure. In some embodiments, the integrity of an analyte is maintained such that it can be accurately quantified in the bodily fluid. Methods for collecting blood or fractions thereof are well known in the art. For example, see U.S. Pat. No. 5,286,262, which is hereby incorporated by reference in its entirety.

A bodily fluid may be obtained from any mammal known, or suspected, to suffer from cardiovascular disease or that can be used as a disease model for cardiovascular disease. In some embodiments, the mammal is a rodent. Examples of rodents include mice, rats, and guinea pigs. In some embodiments, the mammal is a primate. Examples of primates include monkeys, apes, and humans. In some embodiments, the mammal is a human. In some embodiments, the subject has no clinical signs of cardiovascular disease. In other embodiments, the subject has mild clinical signs of cardiovascular disease. In yet other embodiments, the subject may be at high risk for cardiovascular disease. In still other embodiments, the subject has been diagnosed with cardiovascular disease.

Assessment of analyte levels may encompass assessment of the level, amount, or concentration of protein, or the level of enzymatic activity of the protein, wherever applies. In either case, the level is quantified such that a value, an average value, or a range of values is determined. In some embodiments, the level of protein concentration of the cardiovascular disease analyte is quantified. In some embodiments, the concentration of a lipid such as cholesterol or triglyceride is used to represent the VLDL+LDL or HDL component or subtype, e.g. the cholesterol concentration of HDL with apoC-III.

There are numerous known methods and kits for measuring the amount or concentration of a protein or lipid in a sample, including as non-limiting examples, ELISA, western blot, absorption measurement, colorimetric determination, and the like. In certain embodiments, the protein concentration is measured using a luminex based multiplex immunoassay panel. However, it will be understood by those of skill in the art that this disclosure is not limited to any particular assay.

Methods of quantitatively assessing the level of a protein in a biological sample such as plasma are well known in the art. In some embodiments, assessing the level of a protein involves the use of a detector molecule for the analyte. Detector molecules can be obtained from commercial vendors or can be prepared using conventional methods available in the art. Exemplary detector molecules include, but are not limited to, an antibody that binds specifically to the analyte, a naturally-occurring cognate receptor, or functional domain thereof, for the analyte, and a small molecule that binds specifically to the analyte.

In a some embodiments, the level of an analyte is assessed using an antibody. Thus, non-limiting exemplary methods for assessing the level of an analyte in a biological sample include various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1999, Using Antibodies: A Laboratory Manual, Cold Spring Harbor Laboratory Press, NY.

Other methods for assessing the level of a protein include chromatography (e.g., HPLC, gas chromatography, liquid chromatography) and mass spectrometry (e.g., MS, MS-MS). For instance, a chromatography medium comprising a cognate receptor for the analyte or a small molecule that binds to the analyte can be used to substantially isolate the analyte from the biological sample. Small molecules that bind specifically to a analyte can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection.

In some embodiments, the level of enzymatic activity of the analyte if such analyte has an enzymatic activity may be quantified. Generally, enzyme activity may be measured by means known in the art, such as measurement of product formation, substrate degradation, or substrate concentration, at a selected point(s) or time(s) in the enzymatic reaction. There are numerous known methods and kits for measuring enzyme activity. For example, see U.S. Pat. No. 5,654,152. Some methods may require purification of the cardiovascular disease analyte prior to measuring the enzymatic activity of the analyte. In some embodiments, a pure analyte constitutes at least 90%, at least 95% or at least 99% by weight of the analyte in a given sample. Analytes may be purified according to methods known in the art, including, but not limited to, ion-exchange chromatography, size-exclusion chromatography, affinity chromatography, differential solubility, differential centrifugation, and HPLC.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1

### Apolipoprotein C-III as a Potential Modulator of the Association Between HDL-Cholesterol and Incident Coronary Heart Disease

It is herein examined whether the presence or absence of apoC-III differentiates HDL into subtypes with non-protective or protective associations with risk of future CHD.

High-density lipoprotein cholesterol (HDL-C) levels were measured in plasma separated according to apoC-III (by immunoaffinity chromatography) in two prospective case-control studies nested within the Nurses' Health and the Health Professionals Follow-Up Studies. Baseline was in 1990 and 1994, and 634 incident CHD cases were documented through 10 to 14 years of follow-up. The relative risk of CHD per each standard deviation of total HDL-C was 0.78 (95% confidence intervals, 0.63-0.96). The HDL-C subtypes were differentially associated with risk of CHD, HDL-C without apoC-III inversely and HDL-C with apoC-III directly (P=0.02 for a difference between the HDL types). The relative risk per standard deviation of HDL-C without apoC-III was 0.66 (0.53 to 0.93) and 1.18 (1.03 to 1.34) for HDL-C with apoC-III. HDL-C with apoC-III comprised ˜13% of the total HDL-C. Adjustment for triglycerides and apoB attenuated the risks; however, the two HDL-C subgroups remained differentially associated with risk of CHD (P=0.05).

Separating HDL-C according to apoC-III identified two types of HDL with opposing associations with risk of CHD. The results presented herein demonstrate that the proatherogenic effects of apoC-III, as a component of VLDL and LDL, extends to HDL.

Study Design and Population

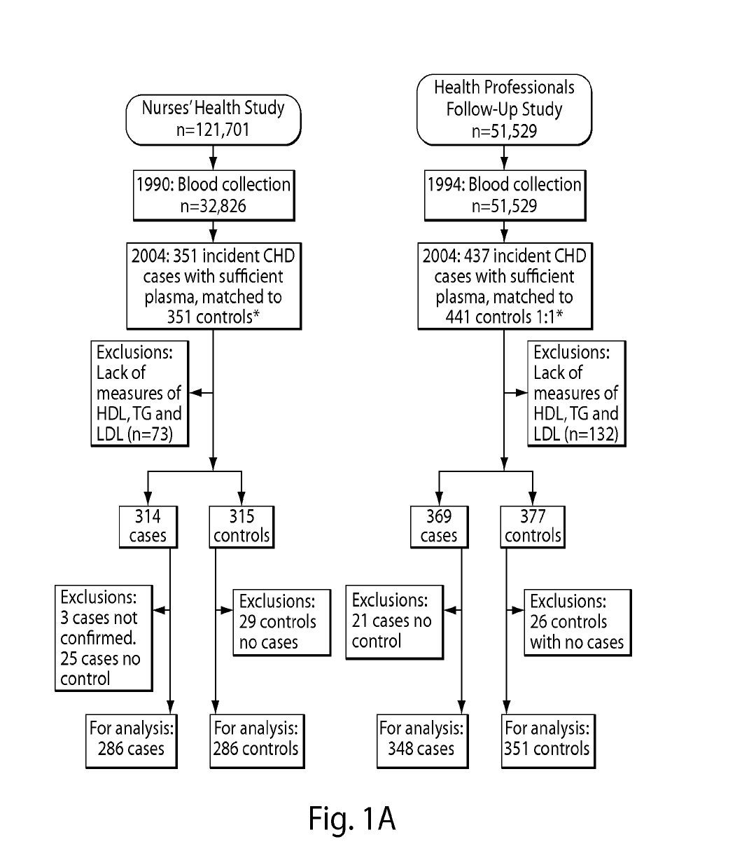

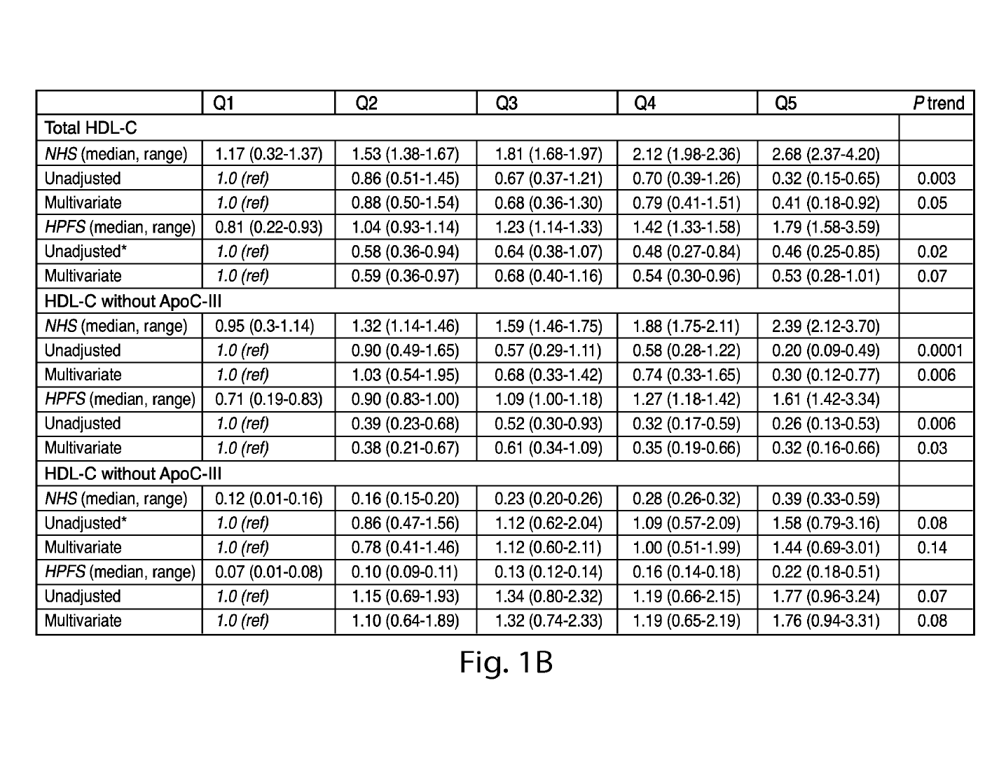

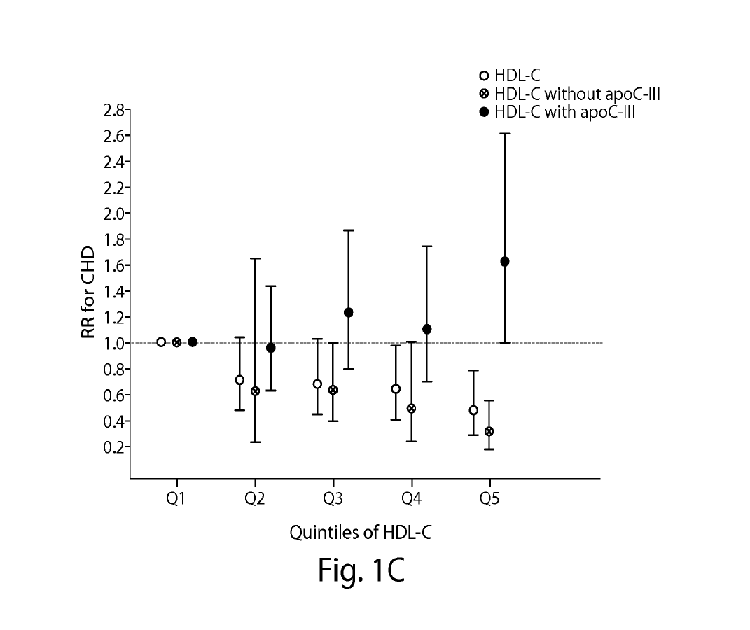

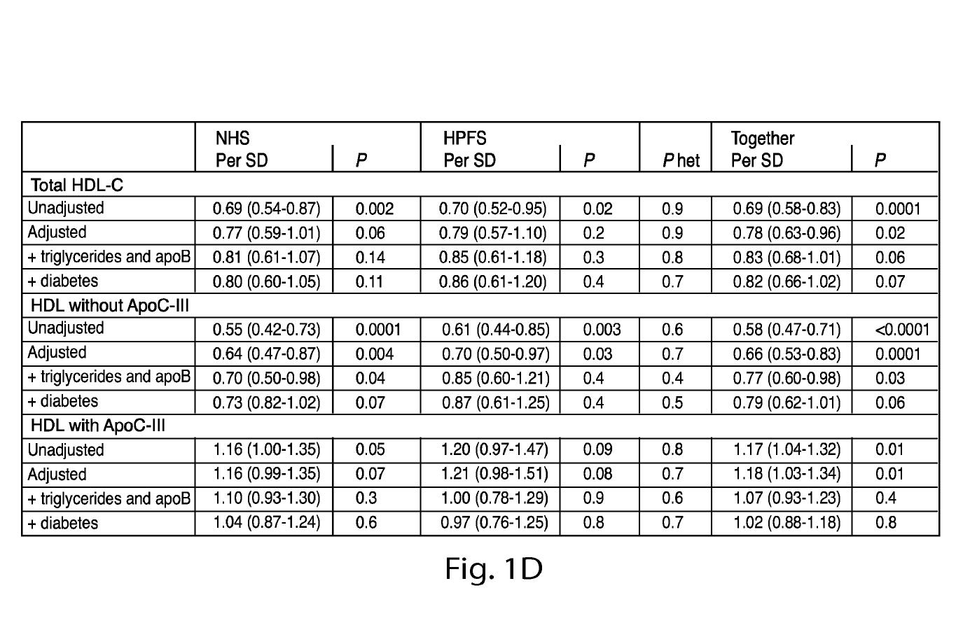

The Nurses' Health Study (NHS) enrolled 121 701 female nurses aged 30 to 55 years in 1976 and the Health Professionals Follow-Up Study (HPFS) enrolled 51 529 males aged 35 to 75 years in 1986. Participants of both cohorts filled out questionnaires on lifestyle and medical history and have since been followed with biennial questionnaires to record newly diagnosed illnesses and to update lifestyle information (Colditz et al., 1997, J Womens Health 6:49-62; Rimm et al., 1992, Am J Epidemiol 135:1114-1126). Between 1989 and 1990, a blood sample was requested from all active participants in NHS and collected from 32 826 women. Similarly, blood samples were requested between 1993 and 1995 and obtained from 18 225 HPFS participants. As described earlier, nested case-control studies of CHD were designed within both cohorts, allowing the study to maintain a prospective design (Pai et al., 2004, N Engl J Med 351:2599-2610). Because the laboratory measurements for the present study required a large volume (0.600 mL), the study was restricted to those with more than 2 mL plasma (FIG. 1A). Among participants who were free of diagnosed cardiovascular disease or cancer at blood draw, 351 women in NHS and 437 men in HPFS with incident CHD between blood draw and June, 2004 were identified. Using risk-set sampling (Prentice et al., 1978, Biometrika 65:153-158), controls were selected randomly and matched in a 1:1 ratio on age (1 year), smoking (never, past, current), and month of blood return, among participants who were free of cardiovascular disease at the time CHD was diagnosed in the case. The diagnosis of CHD included nonfatal myocardial infarction and fatal CHD. The diagnosis of myocardial infarction was confirmed on the basis of the criteria of the World Health Organization (symptoms plus either diagnostic electrocardiographic changes or elevated levels of cardiac enzymes). Deaths were identified from state vital records and the National Death Index or reported by the participant's next of kin or the postal system. Fatal CHD was confirmed by an examination of hospital or autopsy records, by the listing of CHD as the cause of death on the death certificate, if CHD was the underlying and most plausible cause, and if evidence of previous CHD was available.

The study protocol was approved by the institutional review board of the Brigham and Women's Hospital and the Human Subjects Committee Review Board of Harvard School of Public Health.

HDL-C with and without ApoC-III and Risk for Future CHD

Total HDL-C was inversely associated with risk of CHD in both NHS and HPFS (FIG. 1B). An even stronger inverse association was observed across quintiles of HDL-C without apoC-III in both cohorts. The associations appeared slightly stronger in the NHS women than the HPFS men, but the tests of between study heterogeneity for the associations were not significant (P>0.5). When the cohort-specific results from multivariable analyses that adjusted for important demographics and lifestyle factors were combined, the RR across extreme quintiles of HDL-C without apoC-III was 0.31 (95% CI, 0.18-0.55) in the combined cohort (FIG. 1C). Each SD increase (0.53 mmol/L) predicted a RR of 0.66 (95% CI, 0.53-0.83; FIG. 1D). In contrast, higher levels of HDL-C with apoC-III were not inversely associated with the risk of CHD. In the combined cohorts, the RR for quintile 5 versus 1 was 1.62 (CI, 1.00-2.61). Each SD increase in HDL-C with apoC-III (0.07 mmol/L) was associated with an 18% (95% CI, 3-34%) higher risk of future CHD. The slopes of the regression coefficients for the two HDL-C subtypes were statistically significantly different (P=0.02). Additional adjustment for triglycerides and apoB attenuated the risk estimates for HDL-C with apoC-III but the test of heterogeneity for slopes remained statistically significant (P=0.05). After additional adjustment for the potential intermediate exposure, diabetes, the HDL-C with apoC-III was no longer associated with the risk of CHD (RR per SD=1.02 [95% CI, 0.88-1.18]), whereas an inverse trend remained for HDL-C without apoC-III (RR per SD=0.79 [95% CI, 0.62-1.01]; FIG. 1D).

HDL-C with apoC-HI Associated with Higher Risk of Future CHD

The controversies in establishing the role of HDL in atherosclerosis may be due in part to the lack of specificity in the measurements of HDL-C. In two independent prospective studies of generally healthy middle-aged men and women, it was found that HDL is composed of two populations having opposite associations with CHD. The major HDL-C type lacking apoC-III has the expected protective association with CHD, whereas the small subfraction of HDL-C that has apoC-III present on its surface (≈13%) tended to be associated with a higher risk of future CHD.

Investigations of the metabolic heterogeneity of lipid particles are potentially valuable to improve understanding of the atheroprotective or nonprotective effects of HDL. Although previous large-scale studies have evaluated HDL subpopulations separated by size (van der Steeg et al., 2008, J Am Coll Cardiol 51:634-642), whether such measurements improve cardiovascular risk prediction remains uncertain as the findings have been inconsistent (El Harchaoui et al., 2009, Ann Intern Med 150:84-93; Mora, 2009, Circulation 119:2396-2404). Other more experimental sub-classifications include the effect of HDL on cholesterol efflux or anti-inflammatory activities of HDL (Ansell et al., 2003, Circulation 108:2751-2756). These new experimental assays are of scientific interest and suggest that the measure of total HDL-C may be diluted due to a mixing of cholesterol distributed in both anti- and proatherogenic HDL particles. However, so far, the concept and understanding of what makes a dysfunctional or even proinflammatory HDL subtype remains elusive (Movva et al., 2008, Clin Chem 54:788-800; Smith, 2010, Arterioscler Thromb Vasc Biol 30:151-155). The data presented herein suggest that apoC-III may confer atherogenic properties to HDL that potentially could overcome other beneficial components. It was observed that the apoC-III concentration in HDL was not significantly associated with CHD, indicating that the concentration of HDL-C with any apoC-III may be more relevant to the risk of CHD than how much apoC-III is in the HDL. Previous studies have only addressed the latter question. In the CLAS trial, the concentration of apoC-III in HDL was inversely associated with the progression of CAD in the drug-treated group of CAD patients only (Blankenhorn et al., 1990, Circulation 81:470-476), whereas a direct, but not statistically significant, association with CHD and re-current events was reported in two larger studies (Sacks et al., 2000, Circulation 102:1886-1892; Onat et al., 2003, Atherosclerosis 168:81-89). These studies were either cross sectional (Onat et al., 2003, Atherosclerosis 168:81-89) or conducted in a patient population with existing CVD (Sacks et al., 2000, Circulation 102:1886-1892). Because the concentration of apoC-III may be affected by disease status, it may be particularly important to study this in a prospective setting in populations that did not have clinical CVD at baseline. For the metabolism of entire lipoprotein particles, it is likely that the presence (if any) versus absence of apoC-III may determine the downstream interactions with receptors and enzymes (Alaupovic, 1996, Methods Enzymol 263:32-60; Gustafson et al., 1966, Biochemistry 5:632-640). Kawakami et al. (2006, Circulation 113:691-700) reported that HDL without apoC-III, but not HDL with apoC-III, limits the proinflammatory adhesion of human monocytes to endothelial cells. ApoC-III also plays an important role in the catabolism of triglyceride-rich lipoproteins through the inhibition of clearance of plasma VLDL and LDL by the liver (Clayey et al., 1995, Arterioscler Thromb Vasc Biol. 15:963-971; Zheng et al., 2010, Circulation. 121:1722-1734; Sehayek & Eisenberg, 1991, J Biol Chem 266:18259-18267). While not wishing to be bound by any particular theory, it is possible that apoC-III functions similarly in HDL circulating in blood, impairing delivery of HDL-C to the liver. However, without wishing to be bound by any particular theory, it remains a possibility that apoC-III is a marker for other attributes of HDL that are related to atherosclerosis.

Lifestyle factors may modulate the distribution of cholesterol within the two HDL fractions. It was found that alcohol intake was similarly associated with both HDL-C subfractions, whereas body weight and estrogens were only associated with HDL-C without apoC-III. Other unmeasured confounders cannot be excluded.

Earlier studies of apoC-III in lipoprotein fractions have reported concern about redistribution of apoC-III from apoB-containing lipoproteins to HDL during storage (Cohn et al., 2004, J Lipid Res 45:1572-1579). However, in the present study, no instability was detected in the measures of HDL-C with (any) apoC-III and HDL-C without apoC-III in the assessment of samples that were analyzed both fresh and after freezer storage. Use of different methodologies for the separation of the apoC-III-containing lipoprotein fractions may be one explanation.

In conclusion, it was found that HDL-C with and without apoC-III showed opposite associations with the risk of CHD in prospective studies of apparently healthy men and women. On adjustment for a proatherogenic lipid profile and diabetes, HDL-C with apoC-III was no longer associated with risk of CHD, but there was no evidence for an inverse association. The findings presented herein highlight that HDL comprises a group of particles that may be more or less closely linked with atherosclerosis. HDL that has apoC-III may represent a dysfunctional HDL lacking its cardioprotective function. This may also have implications for future development novel therapeutic interventions aimed at HDL elevation, as the cardioprotective benefits may differ depending on the affected HDL subfraction.

### Example 2

### Apolipoprotein E in VLDL and LDL Mitigates the Risk of Coronary Heart Disease Associated with Apolipoprotein C-III

Experiments were designed to explore whether apoE may attenuate the atherogenicity of apoC-III by examining the association between apoE concentrations in VLDL and LDL with apoC-III and coronary heart disease (CHD). This relationship was prospectively studied in two large US cohorts of women and men, the Nurses' Health Study (NHS) and the Health Professionals Follow-up Study (HPFS). The association between apoE content (number of molecules per particle) in VLDL and LDL that have apoC-III, and coronary heart disease (CHD) was investigated. Three-hundred and twenty-two women and 418 men initially free of cardiovascular disease who developed a fatal or nonfatal myocardial infarction during 10 to 14 years of follow-up, and matched controls who remained free of CHD were studied. The apoE content of VLDL with apoC-III was inversely associated with CHD after multivariable adjustment (relative risk for top versus bottom quintile 0.52, 95% CI 0.34-0.79). The apoE content of LDL with apoC-III had a similar inverse association with CHD. The molar ratio of apoE to apoC-III in LDL was also inversely associated with risk in multivariable models (relative risk 0.55, 95% CI 0.35-0.88). Higher LDL apoE contents were protective at different levels of LDL apoC-III. The observed associations were independent of traditional CHD risk factors and of C-reactive protein.

The data presented herein show that high contents of apoE in VLDL or LDL with apoC-III predicted a lower risk of CHD. In LDL, apoE mitigates the increased risk associated with apoC-III. Strategies to enrich LDL in apoE are worth exploring for the prevention of CHD.

Association of apoE in LDL and VLDL with apoC-III and CHD

The average content of apoE (as reflected by the apoE:apoB molar ratio) in LDL with apoC-III was significantly lower in cases than in matched controls of both sexes (FIG. 3). Meanwhile, the apoE content of VLDL with apoC-III was not different in cases and controls.

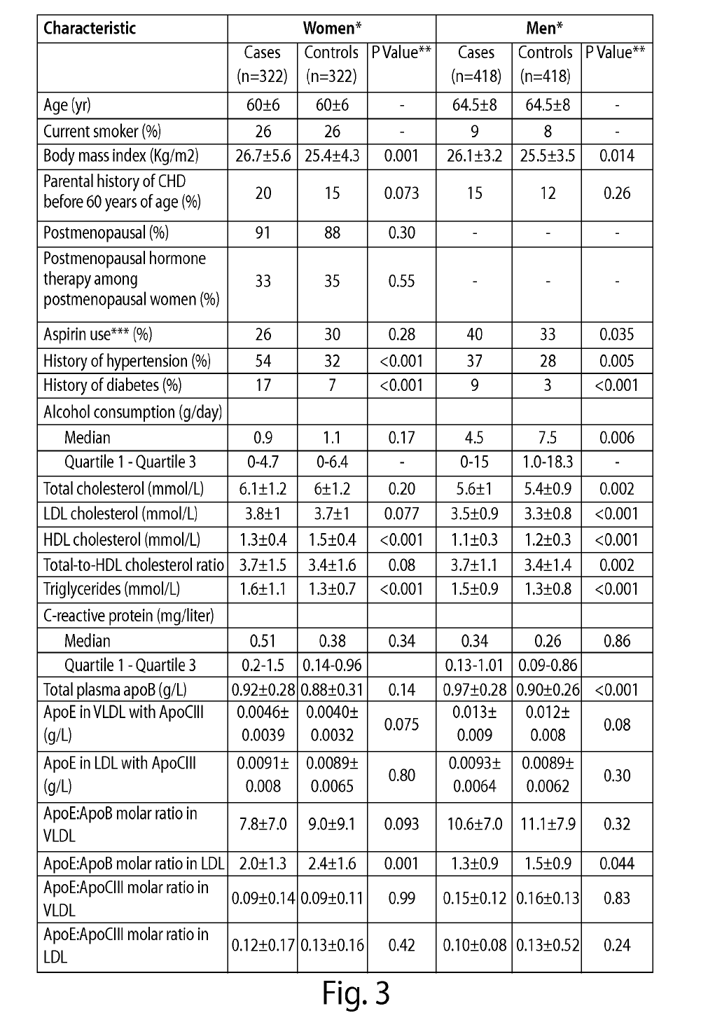

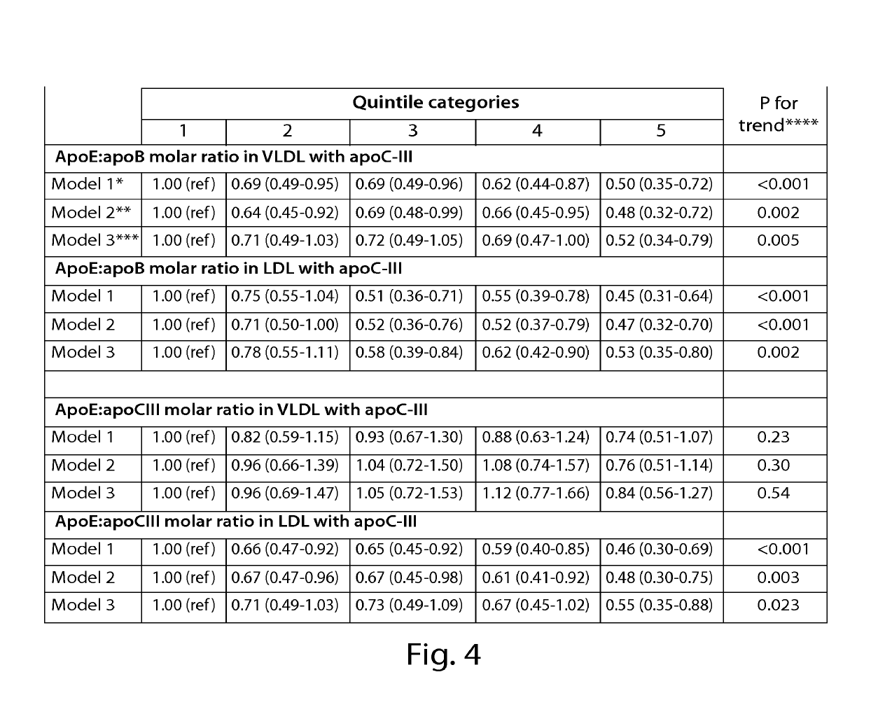

The apoE content per each VLDL with apoC-III, expressed as the apoE:apoB molar ratio in this fraction, showed a strong negative association with CHD (relative risk for top versus bottom quintile 0.50, 95% CI 0.35-0.72; P for trend <0.001) that persisted after additional multivariable adjustment in models 2 and 3 (FIG. 4). The same result was evident for LDL with apoC-III, higher apoE content per particle predicted a markedly reduced risk (relative risk 0.45, 95% CI 0.31-0.64) that continued to be significant after multivariable adjustment. The relative abundance of apoE respect to apoC-III in LDL (expressed as the apoE:apoC-III molar ratio) showed a strongly negative association with CHD in all three models (FIG. 4). The apoE:apoC-III molar ratio in VLDL showed no association with CHD. The plasma concentration of apoE in VLDL with apoC-III showed a trend towards a positive association with CHD (relative risk 1.45, 95% CI 0.98-2.17 in model 3), probably because apoC-III and apoE concentrations in VLDL were correlated (r=0.63). The plasma concentration of apoE in LDL with apoC-III was not associated with risk (relative risk 1.33, 95% CI 0.87-2.04 in model 3), concordant with its correlation with apoC-III being less strong than in VLDL (r=0.45).

In order to establish if the attenuation of risk by apoE in LDL happened at different levels of apoC-III, participants were classified in joint tertiles of apoE and apoC-III content per LDL particle. A higher apoE content in LDL was associated with lower risk across all tertiles of apoC-III content in LDL with no significant interaction (P value for interaction=0.42), suggesting that apoE in LDL effectively antagonizes apoC-III even when it is abundant.

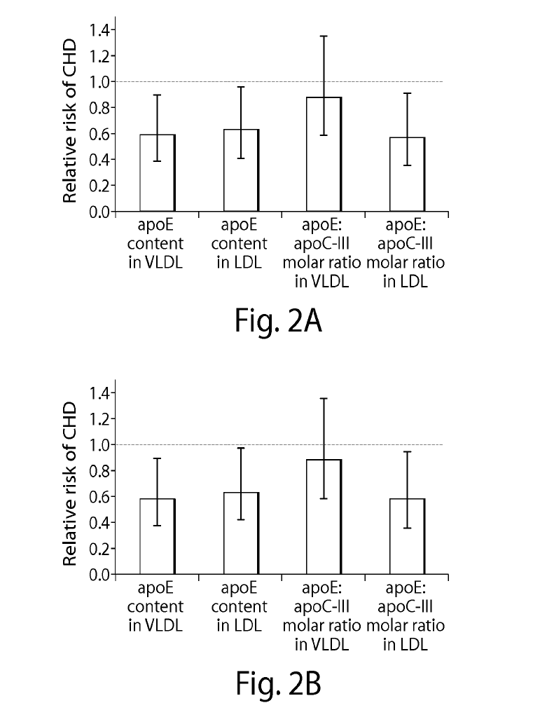

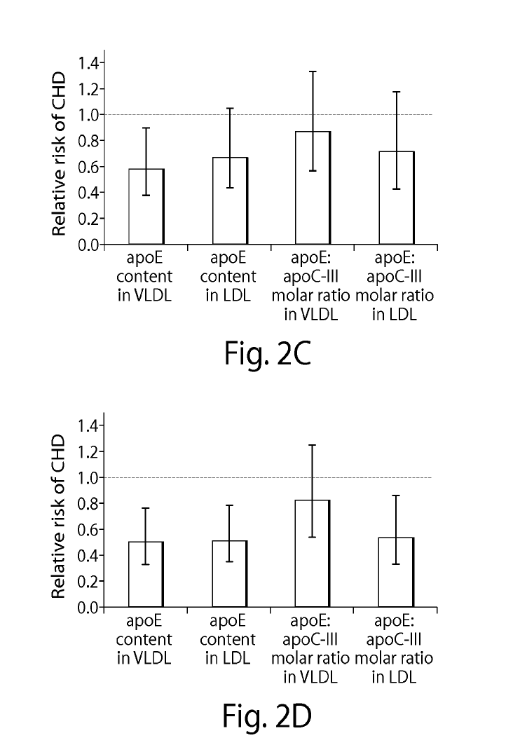

Adjustment for plasma LDL cholesterol in the background of multivariable model 3 only partially attenuated the negative association between apoE content in VLDL that has apoC-III and CHD (relative risk 0.61, 95% CI 0.40-0.93) and the association between apoE content of LDL that has apoC-III and CHD (relative risk 0.62, 95% CI 0.41-0.93). The inverse association for the apoE:apoC-III ratio in LDL was also not affected by plasma LDL adjustment (relative risk 0.59, 95% CI 0.37-0.95). The same pattern was observed in multivariable models adjusted for plasma HDL cholesterol or plasma triglycerides, the association for both apoE content of VLDL with apoC-III and apoE content in LDL with apoC-III were only partially attenuated. Given that apoE has been proposed to have anti-inflammatory properties, multivariable analyses that included baseline plasma C-reactive protein (CRP) levels were performed. In these models, the apoE content of VLDL with apoC-III (relative risk 0.51, 95% CI 0.33-0.77), the apoE content of LDL with apoC-III (relative risk 0.52, 95% CI 0.34-0.78) and the apoE:apoC-III ratio in LDL (relative risk 0.56, 95% CI 0.35-0.89) were still negatively associated with CHD (FIG. 2).

Finally, to explore the impact of LDL apoE content in a scenario of constant particle number, a logistic model that included concentrations of apoB, apoC-III and apoE in LDL with apoCIII was run. In this model, the apoE content of LDL was still negatively associated with CHD risk (relative risk 0.69, 95% CI 0.45-1.07).

apoE Content Relative to apoC-III in LDL is Protective Against CHD

The results from this prospective study strongly suggest that apoE reduces the atherogenicity of LDL that contains apoC-III, and that the abundance of apoE relative to apoC-III in LDL is a protective factor against CHD. The apoE content of VLDL also exhibited a negative association with CHD, but the balance between apoE and apoC-III appeared to be less relevant for this lipoprotein type. The protective effect of apoE in LDL appeared to be independent of plasma LDL cholesterol levels, and to persist even when apoC-III levels in LDL are high.

Even though this finding makes sense given the biological antagonism between the two apolipoproteins and the well documented antiatherogenic properties of apoE (Curtiss, 2000, Arterioscler Thromb Vasc Biol 20:1582-53), the influence of apoE in lipoproteins on hard cardiovascular endpoints is very scantly documented. The results presented herein also emphasize the increasingly recognized importance of the composition and physicochemical properties of lipoproteins as more refined and often more relevant variables in the exploration of pathophysiological mechanisms than the total concentrations of plasma lipids. Even though only 10-20% of LDL contain apoC-III (Campos et al., 2001, J Lipid Res 42:1239-1249), the idea that apoE can effectively protect against the risk provided by these particles is very appealing if one considers that LDL with apoC-III are very strongly associated with CHD (Lee et al., 2003, Arterioscler Thromb Vasc Biol 23:853-858), and that in fact much of the CHD risk ordinarily attributed to total LDL may be due to this subpopulation that contains apoC-III (Mendivil et al., 2011, Circulation 124:2065-2072).

Prior studies in older adults have found a positive association between total plasma apoE levels and CVD mortality (Mooijaart et al., 2006, PLoS Med 3(6):e176), or the risk of stroke (van Vliet et al., 2007, Ann N Y Acad Sci 1100:140-7). From the results presented herein, it is apparent that the distribution of total plasma apoE across the different lipoprotein fractions can greatly affect its association with CVD. In this respect, an observational analysis of patients from the Cholesterol and Recurrent Events (CARE) trial found a positive association between apoE in HDL and the risk of coronary events (Sacks et al, Circulation 2000; 102:1886-1892; the entire contents of each of which are incorporated herein by reference). This might explain the positive association between plasma apoE and CVD in some studies, given that about 40-50% of apoE is found on HDL (Luc et al., 1996, J Lipid Res 37:508-517). Nevertheless, the results presented herein suggest that the relatively minor fraction of plasma apoE that is found in LDL may have a remarkable impact on the risk of CHD. One study in two European countries found higher plasma apoE concentrations in VLDL+LDL of myocardial infarction survivors relative to controls (Mahley & Rall, 2000, Annu Rev Genomics Hum Genet 1:507-37). This could be due to the high correlation between apoE and apoB, as apoB represents the particle concentration of VLDL and LDL and is a well-established CVD risk factor. In the present study, when the variation in apoB levels is excluded by normalizing apoE to the apoB content per particle, apoE was clearly protective in both VLDL and LDL. Also, the absence of an effect modification after adjustment for CRP levels suggests that the anti-inflammatory properties of apoE in LDL do not have a major repercussion on its apparent protective ability against CHD.

### Example 3

### Low-Density Lipoproteins Containing Apolipoprotein C-III and the Risk of Coronary Heart Disease

The association between plasma LDL with apoC-III and coronary heart disease was studied in 320 women and 419 men initially free of cardiovascular disease who developed a fatal or nonfatal myocardial infarction during 10 to 14 years of follow-up and matched controls who remained free of coronary heart disease. Concentrations of LDL with apoC-III (measured as apoB in this fraction) were associated with risk of coronary heart disease in multivariable analysis that included the ratio of total cholesterol to high-density lipoprotein cholesterol, LDL cholesterol, apoB, triglycerides, or high-density lipoprotein cholesterol and other risk factors. In all models, the relative risks for the top versus bottom quintile of LDL with apoC-III were greater than those for LDL without apoC-III. When included in the same multivariable-adjusted model, the risk associated with LDL with apoC-III (relative risk for top versus bottom quintile, 2.38; 95% confidence interval, 1.54-3.68; P for trend <0.001) was significantly greater than that associated with LDL without apoC-III (relative risk for top versus bottom quintile, 1.25; 95% confidence interval, 0.76-2.05; P for trend=0.97; P for interaction <0.001). This divergence in association with coronary heart disease persisted even after adjustment for plasma triglycerides.

To evaluate whether plasma concentrations of VLDL and LDL that have apoC-III, as measured by apoB in each of them, are more strongly associated with CHD risk than those of the same lipoproteins without apoC-III, two US populations initially free of CHD were prospectively studied: the Nurses' Health Study (NHS; women) and the Health Professionals Follow-up Study (HPFS; men).

These results suggest that the concentration of LDL with apoC-III is independently associated with the risk of CHD and that much of the risk ordinarily attributed to LDL may in fact be due to LDL particles that contain apoC-III. These findings are consistent with prior in vitro, animal, and epidemiological evidence suggesting enhanced atherogenicity of lipoproteins containing apoC-III. More than contributing to the improvement of already excellent prediction models (Wilson et al., 1998, Circulation 97:1837-1847; Assmann et al., 2002, Circulation 105:310-315, Ridker et al., 2007, JAMA 297:611-619), these results may endorse LDL with apoC-III as a highly attractive target for the prevention or treatment of atherosclerotic diseases.

Risk Associated with LDL with and without apoC-III

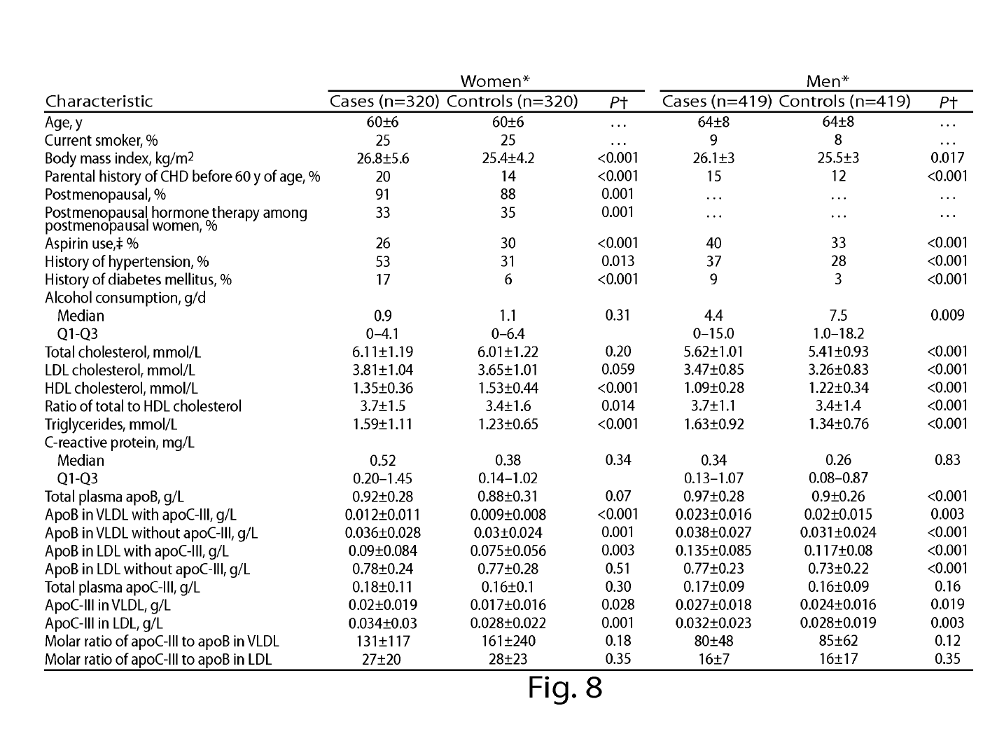

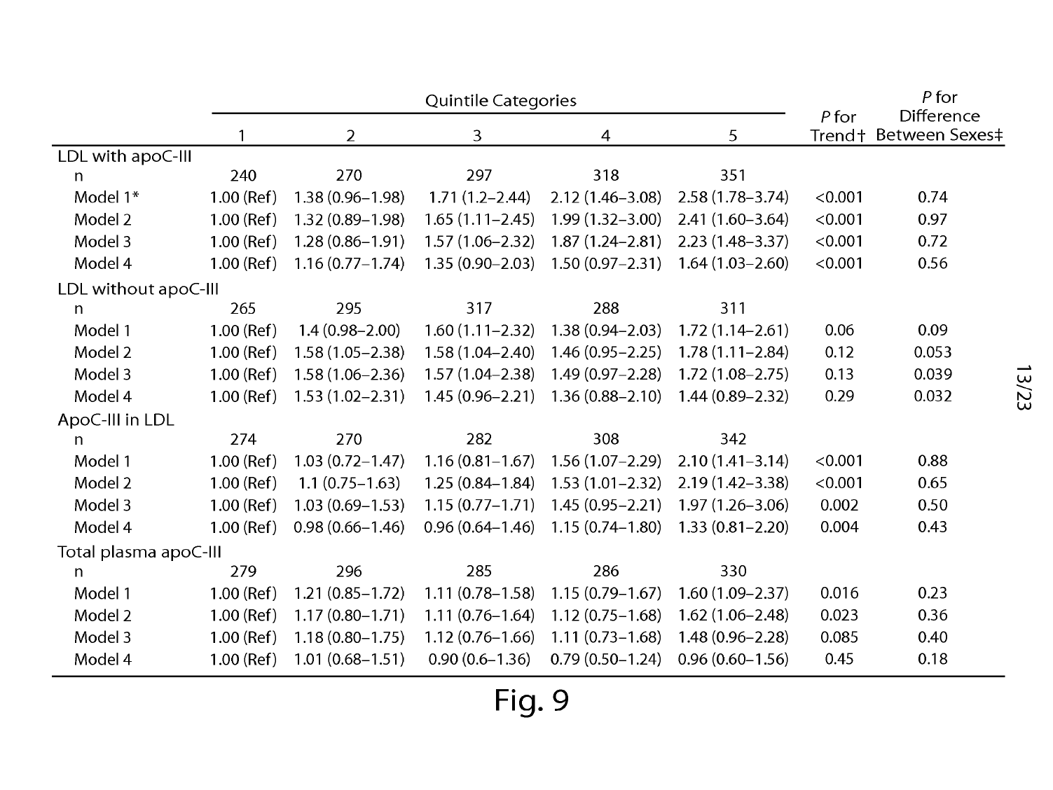

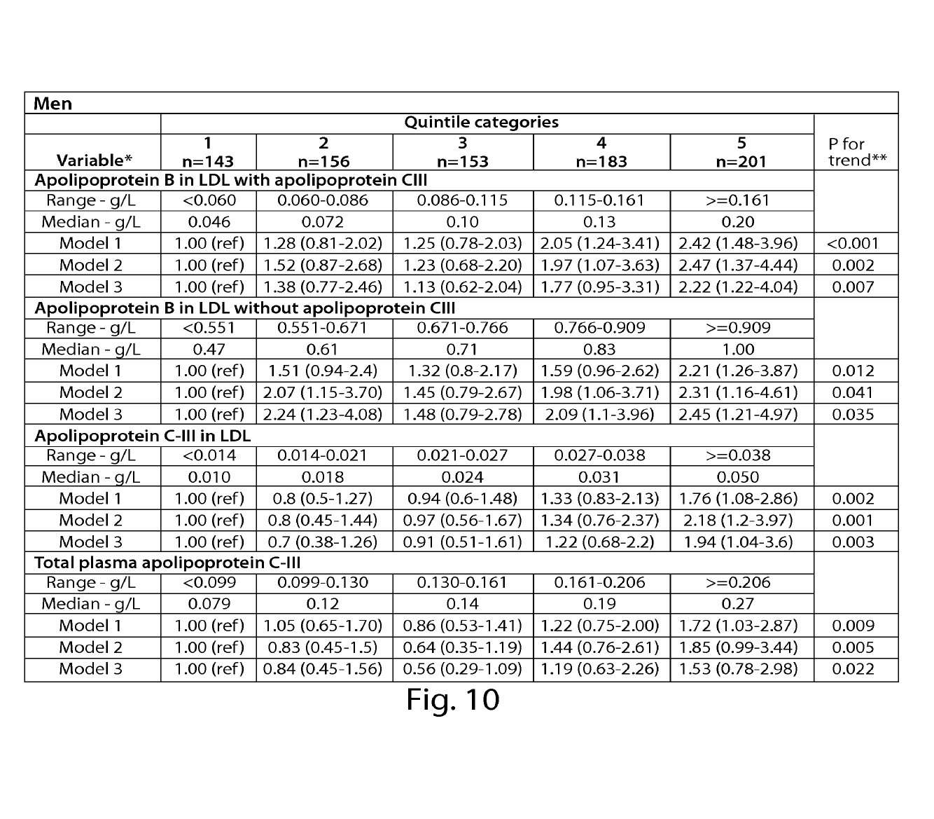

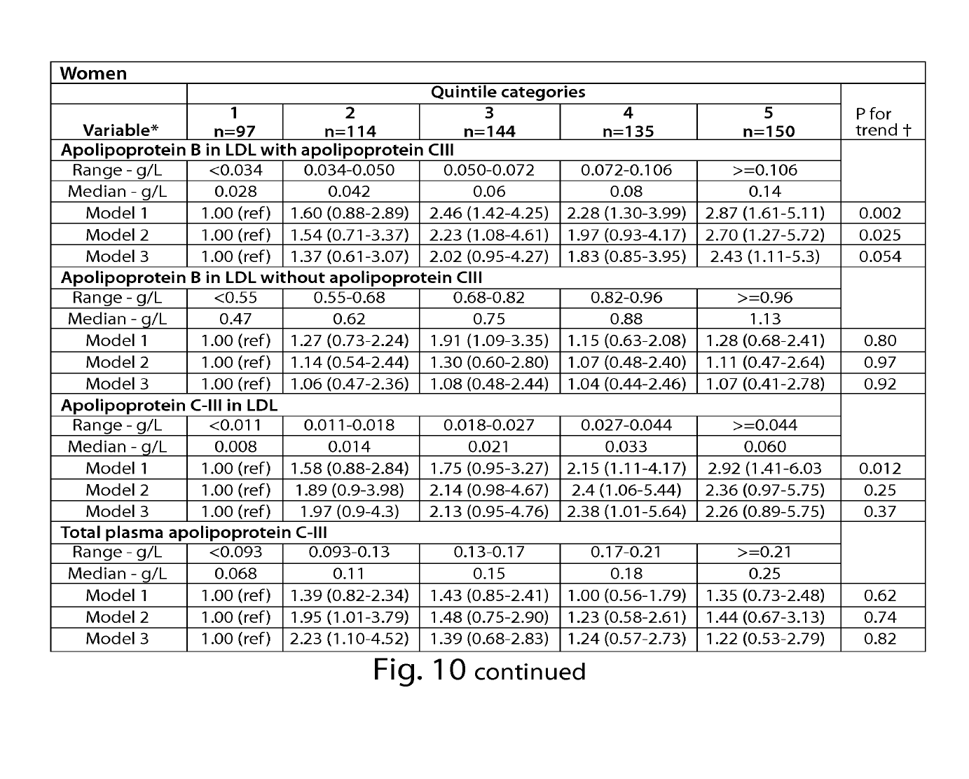

LDL with apoC-III represented on average 12% of total LDL. Participants of both sexes who developed CHD during follow-up had significantly higher concentrations of LDL with apoC-III. Male but not female cases had higher concentrations of LDL without apoC-III than controls (FIG. 8). Compared with participants in the lowest quintile, participants in the highest quintile of LDL with apoC-III had a significantly increased risk of CHD (relative risk for highest versus lowest quintile, 2.58; 95% confidence interval, 1.78-3.74; P for trend <0.001), conditioning in matching factors (FIG. 9). LDL without apoC-III was associated with CHD but with a lower relative risk than LDL with apoC-III (1.72; 95% confidence interval, 1.14-2.61). Adjustment for other risk factors in models 2 through 4, including personal history of diabetes mellitus and triglyceride concentration, had little effect on the relative risks (FIG. 9). The risk associated with LDL with apoC-III was similar in NHS and HPFS participants, with no significant sex interaction, but the risk associated with LDL without apoC-III was higher in HPFS (FIG. 10). It was not possible to perform multivariate logistic analyses in the diabetic subgroup (n=126) because of its small size (model did not converge), but among nondiabetic participants (n=1350), the multivariate-adjusted results were consistent with those for the complete study sample (relative risk for top versus bottom quintile of LDL with apoC-III, 2.39; 95% confidence interval, 1.56-3.67).

Independence of Association of LDL with apoC-III from Other Lipid Risk Factors

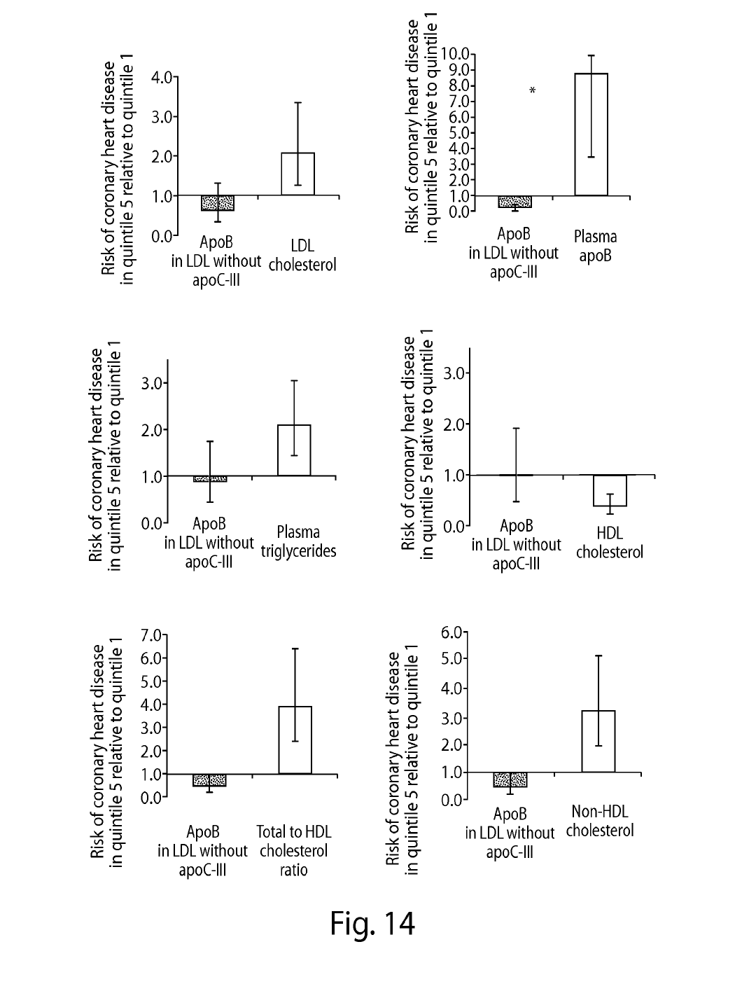

Whether the association between LDL with apoC-III and CHD was independent of other established lipid risk factors was explored. In the background of model 2, LDL cholesterol, HDL cholesterol, plasma apoB, plasma triglycerides, ratio of total cholesterol to HDL cholesterol, or non-HDL cholesterol were added, one by one; in all models, LDL with apoC-III remained significantly associated with CHD (FIG. 5). In contrast, LDL without apoC-III was not significantly independent of LDL cholesterol, plasma triglycerides, ratio of total cholesterol to HDL cholesterol, or non-HDL cholesterol (FIG. 14).

No Effect Modification by Hypertriglyceridemia

To address the strength of the relationship between LDL with apoC-III and CHD at different triglyceride levels, first a quintile interaction term of triglyceridesxLDL with apoC-III was added to the model, and its coefficient was not significant (P=0.46). Second, the relative risk for LDL with apoC-III was computed in the participants who had normal or high triglycerides according to the standard cut point, 150 mg/dL. The point estimates of the relative risks were similar: 1.61 for participants with triglycerides <150 mg/dL and 1.77 for participants with triglycerides >150 mg/dL (P for interaction=0.63). Thus, the finding regarding LDL with apoC-III as an independent predictor of CHD was not significantly modified by hypertriglyceridemia.

Comparison of LDL with and without apoC-III

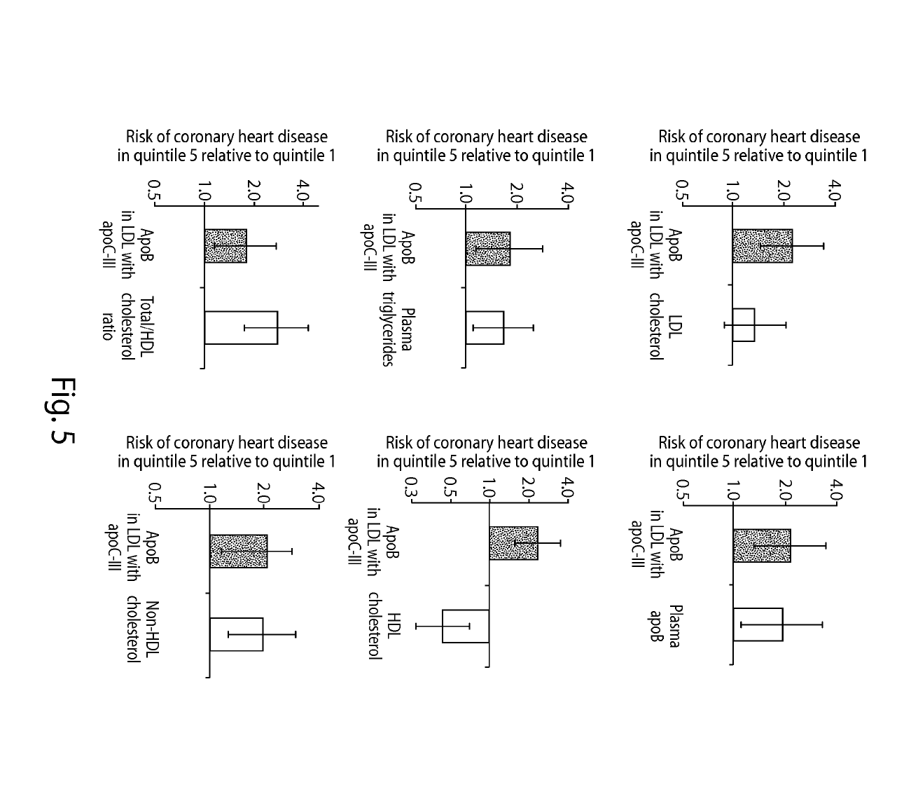

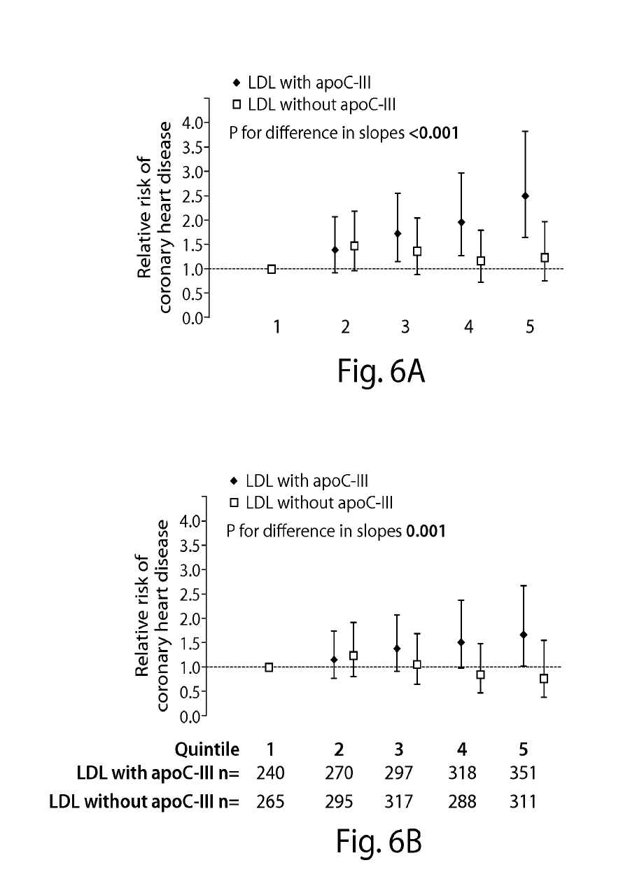

When quintiles of LDL with apoC-III and quintiles of LDL without apoC-III were included in the same multivariable-adjusted model (FIG. 6A), only LDL with apoC-III was associated with CHD (relative risk, 2.38; 95% confidence interval, 1.54-3.68; versus relative risk, 1.25; 95% confidence interval, 0.76-2.05 for LDL without apoC-III; P for difference in slopes <0.001). Given that apoC-III stimulates hepatic secretion of triglycerides, additional analyses were performed adding plasma triglycerides to this model (FIG. 6B). The association with CHD was still significantly higher for LDL with apoC-III (P for difference in slopes=0.001).

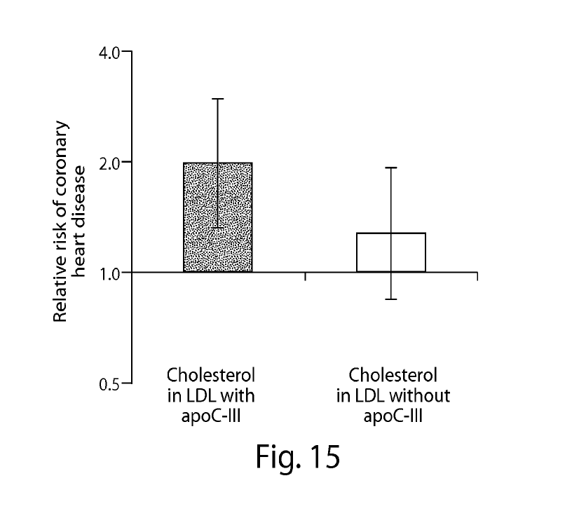

Similar results were obtained in a model adjusted for the same covariates in which cholesterol substituted for apoB. Cholesterol in LDL with apoC-III (relative risk for highest versus lowest quintile, 2.01; 95% confidence interval, 1.35-2.99), but not cholesterol in LDL without apoC-III (relative risk for highest versus lowest quintile, 1.30; 95% confidence interval, 0.86-1.95), was significantly associated with CHD (FIG. 15).

Analyses for apoC-HI Concentrations

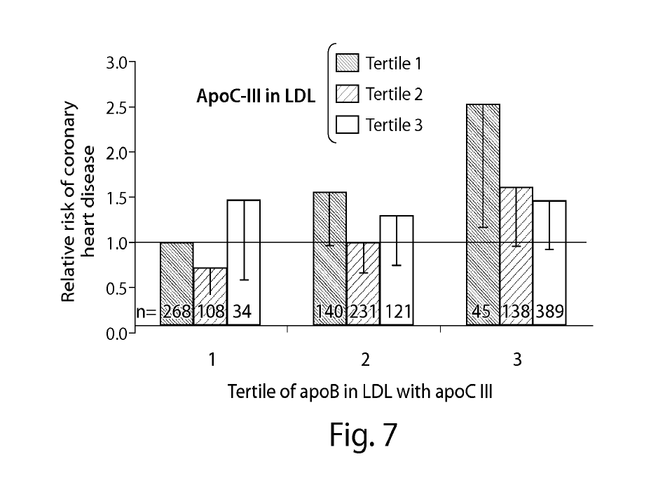

The concentration of apoC-III in LDL was associated with CHD after adjustment for risk factors (FIG. 9), but the association was less consistent than for the apoB concentration of LDL with apoC-III (concentration of LDL particles with any apoC-III; FIG. 7). Total plasma apoC-III was associated with increased risk of CHD (FIG. 9), but the association lost significance in model 3 or after addition of any of the major known lipid risk factors to model 2.

Analyses of VLDL

Concentrations of VLDL with apoC-III and VLDL without apoC-III were associated with CHD similarly and significantly. Adjustment for body mass index and diabetes mellitus in model 3 attenuated the relative risks for both VLDL types, but both were still significant (FIG. 11).

Relative Size of VLDL and LDL with and without apoC-HI

The mean molar ratio of triglycerides to apoB, an indicator of VLDL particle size, was higher for VLDL with apoC-III than for VLDL without apoC-III (11 724 versus 7988) in the study sample. The mean molar ratio of cholesterol to apoB, an indicator of LDL particle size, was higher for LDL with apoC-III than for LDL without apoC-III (2934 versus 2373).

Characteristics Associated with a High Concentration of LDL with apoC-HI

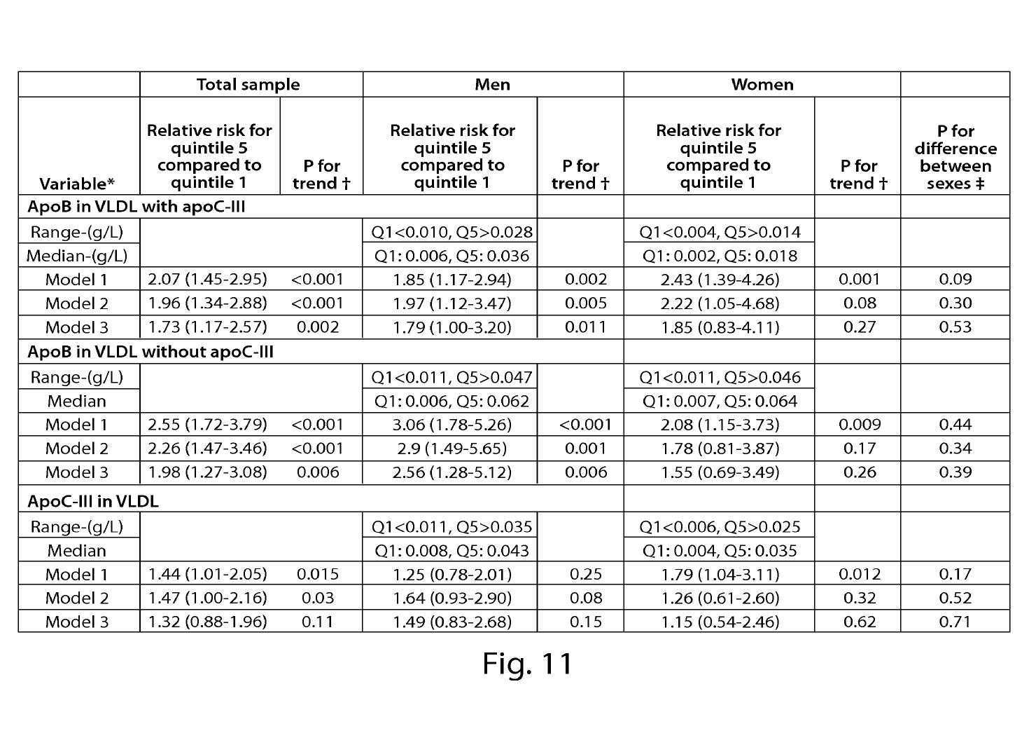

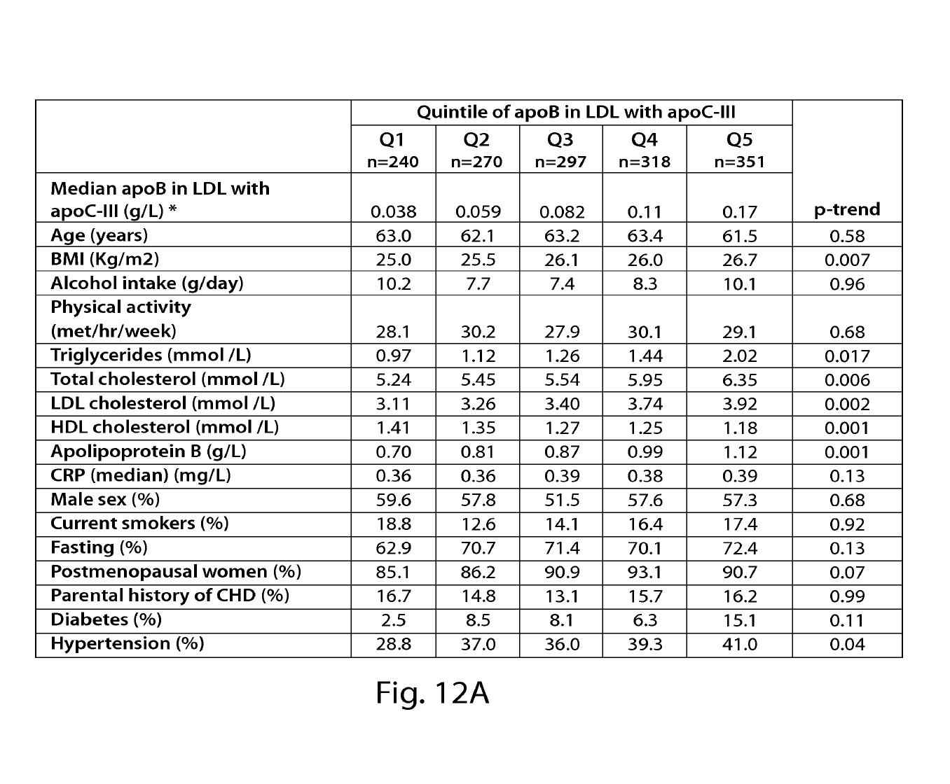

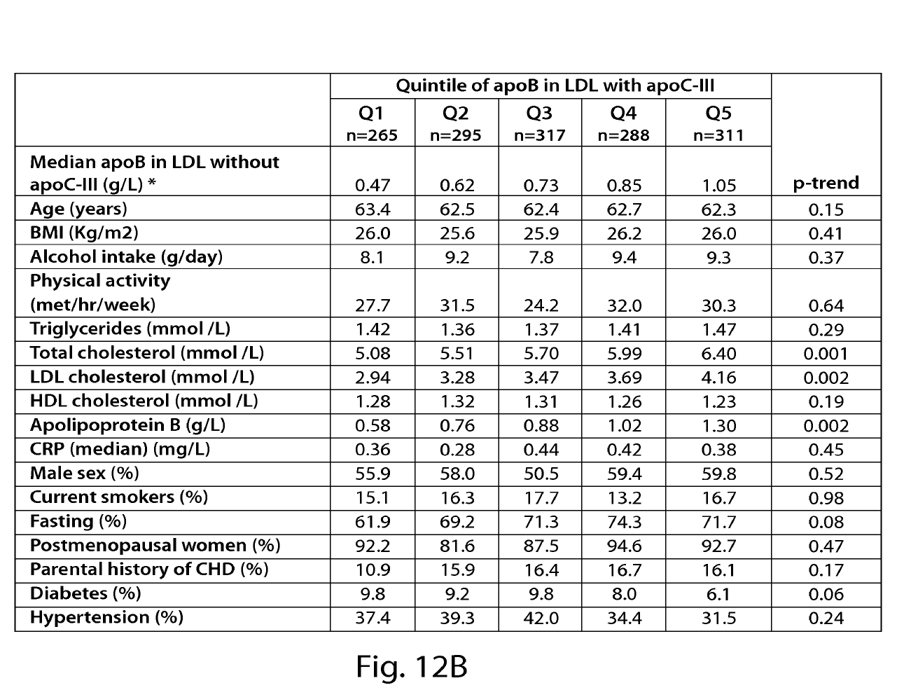

Higher levels of LDL with apoC-III were associated with a markedly higher prevalence of diabetes mellitus, higher plasma triglycerides, lower HDL cholesterol levels, slightly higher body mass indexes, and a higher prevalence of hypertension (FIG. 12). Contrastingly, higher levels of LDL without apoC-III were not associated with any of these conditions but were associated with higher LDL cholesterol and plasma apoB as was LDL with apoC-III.

Analyses Excluding Statin Users

In analyses restricted to the 704 participants who never received cholesterol-lowering medications during follow-up and adjusted for all variables in model 2, the relative risk of CHD for the top versus bottom quintile of LDL with apoC-III was 2.6 (95% confidence interval, 0.87-7.83; P for trend=0.049).

Use of Postmenopausal Hormone Replacement Therapy and LDL Types

To explore whether hormone replacement therapy might confound the association between LDL with apoC-III and CHD, levels of LDL with and without apoC-III were compared among women who used or did not use hormone replacement. Levels of LDL with apoC-III did not differ between users and nonusers (0.85 mg/dL for users versus 0.81 mg/dL for nonusers; P=0.53). The same was observed for LDL without apoC-III (77.6 mg/dL for users versus 77.5 mg/dL for nonusers; P=0.96).

Association of LDL that Contains apoC-III and CHD

In this study of individuals initially free of cardiovascular disease, the concentration of LDL that contains apoC-III was robustly associated with CHD, significantly more so than LDL that does not contain apoC-III.

The association between LDL with apoC-III and CHD was linear and persisted after adjustment for diverse risk factors. In fact, the association was only slightly affected by adjustment for alcohol use, family history of CHD, diabetes mellitus, hypertension, or hormone use, although the study had limited power to address subtle modification. When concentrations of LDL with apoC-III and LDL without apoC-III were included in the same model, only LDL with apoC-III was associated with CHD, suggesting that a considerable proportion of the CHD risk commonly attributed to LDL concentrations is in fact due to one of its subfractions that contains apoC-III. This result extends previous finding in patients with preexisting CHD and type 2 diabetes mellitus (Lee et al., 2003 Arterioscler Thromb Vasc Biol. 23:853-858). The sample size of this study, 739 cases, was able to identify a minimum increased relative risk of 1.4 for either type of LDL. Although not wishing to be bound by any particular theory, it is possible that a larger sample size would identify a mild relation between LDL without apoC-III and CHD.

Besides its effects that impair lipoprotein metabolism and stimulate monocyte adhesion to vascular endothelial cells, apoC-III may promote the inflammatory process that fuels atherosclerosis through activation of Toll-like receptor 2 in monocytes (Kawakami et al., 2008, Circ Res 103:1402-1409; Kawakami et al., 2007, Arterioscler Thromb Vasc Biol 27:219-225) and through induction of insulin resistance and inflammatory signaling pathways governed by nuclear factor-KB in endothelial cells (Kawakami et al., 2006, Circulation 114:681-687; Kawakami et al., 2008, Circulation. 118:731-742). Finally, heterozygote carriers of a nonsense mutation in the apoC-III gene have significantly less coronary artery calcification and CHD than noncarrier subjects from the same population (Pollin et al., 2008, Science 322:1702-1705). All this evidence provides biological plausibility for a direct involvement of LDL with apoC-III in atherosclerosis.

There are on average 10 to 20 molecules of apoC-III but only 1 molecule of apoB in each LDL with apoC-III (Campos et al., 2001, J Lipid Res 42:1239-1249). The apoB concentration of LDL with apoC-III was more strongly associated with CHD than the apoC-III concentration of the same LDL. Thus, the number of apoC-III molecules per LDL may be less important than the concentration of this type of LDL. This was not what was found for VLDL, in which particles with or without apoC-III were similarly associated with CHD risk. Thus, the biological effects of apoC-III may be lipoprotein specific. In this respect, enhancement of LDL adhesion to proteoglycans by apoC-III depends on a critical site in apoB (Hiukka et al., 2009, Diabetes 58: 2018-2026). Although not wishing to be bound by any particular theory, it is conceivable that this site may be exposed in LDL only after most triglycerides in VLDL have been lipolyzed.

Although not wishing to be bound by any particular theory, these results suggest only a modest association between total plasma levels of apoC-III and CHD that was attenuated after adjustment for other lipid risk factors. Plasma apoC-III is a relatively simple determination in a clinical laboratory, whereas the measurement of LDL with apoC-III is more complex and time-consuming but seems to provide more meaningful information.

The association between LDL with apoC-III and CHD was independent of LDL cholesterol, in agreement with previous evidence showing that plasma VLDL and LDL with apoC-III predict the progression of atherosclerosis even among patients whose LDL cholesterol is markedly reduced with lovastatin (Alaupovic et al., 1997, Arterioscler Thromb Vasc Biol 17:715-722). Thus, LDL with apoC-III may explain part of the residual CHD risk among individuals without elevated LDL cholesterol.

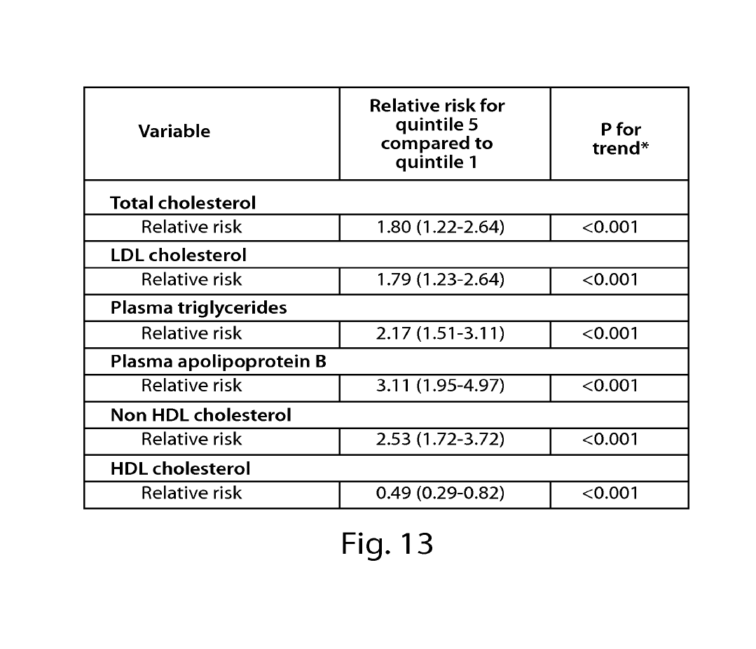

These analyses excluding users of cholesterol-lowering drugs at any point during follow-up showed that, among never users, the relative risk estimate for LDL with apoC-III was quite similar to that in the complete study sample, ruling out confounding by indication of statins. This result is in accordance with a published analysis of the impact of pravastatin on concentrations of lipoproteins with and without apoC-III in patients with diabetes mellitus from the Cholesterol and Recurrent Events (CARE) trial (Lee et al., 2003, Am J Cardiol 92:121-124): Statin treatment reduced LDL with apoC-III by 29% and LDL without apoC-III by 30%. In addition, all traditional lipid risk factors were associated with CHD in the study sample (FIG. 13), so the results for LDL with apoC-III do not seem to be the consequence of an unusual population.

The indirect estimation of LDL size by the molar ratio of cholesterol to apoB showed that LDL with apoC-III was on average larger than LDL without apoC-III, so it seems unlikely that LDL with apoC-III is just acting as a proxy for small, dense LDL. ApoC-III has been reported to transfer from VLDL to HDL during freezing or storage (Cohn et al., 2004, J Lipid Res 45:1572-1579). In this regard, a small experiment was performed in samples from 4 volunteers measuring apoC-III in VLDL, LDL, and HDL immediately after plasma separation and after 5 months of storage at −80° C. The distribution of apoC-III changed only very slightly and nonsignificantly (from 14% in VLDL, 16% in LDL, and 70% in HDL when analyzed fresh to 12% in VLDL, 15% in LDL, and 73% in HDL after freezing/storage/thawing), and although not wishing to be bound by any particular theory, it is believed that this effect is unlikely to have had an important influence on the results under the sample handling and preserving procedures. Another potential concern is whether a high concentration of LDL with apoC-III might just be identifying subjects with hypertriglyceridemia and mild insulin resistance. Although there was a significant correlation between LDL with apoC-III and plasma triglycerides (r=0.42), the association between CHD and LDL with apoC-III persisted after adjustment for triglycerides, suggesting that both characteristics may have common origins but are independently associated with CHD.

### Example 4

### The Concentration of Apolipoproteins C-I and C-II on LDL Particles and Risk of CHD in Prospective Studies of Women and Men

Experiments were designed to examine whether the concentrations of apoC-I and apoC-II in LDL with and without apoC-III would play an independent role in the association with risk of CHD.

Briefly, LDL was isolated by ultracentrifugation from plasma already separated by presence or absence of apoC-III in 568 CHD cases and controls from two parallel nested case-control studies in the Nurses' Health and Health Professionals Follow-up Studies. The concentration of ApoC-I and apoC-II were assessed in both sub-fractions.

In multivariable logistic regression analyses, both apoC-I and apoC-II in LDL were directly associated with risk of CHD, apoC-II more so than apoC-I. ApoC-II, but not apoC-I, was robust to adjustment for apoC-III in LDL. The RR associated with high levels of apoC-II was found especially on LDL particles that also had apoC-III (RR; Q5 vs Q1=3.7; 2.1-6.7; p trend=0.002) and this strong direct association persisted when adjusted for apoC-III in LDL. In contrast, apoC-II in LDL without apoC-III was not significantly associated with a higher risk when adjusted for apoC-III in LDL (RR; Q5 vs Q1=0.9; 0.4-1.8).

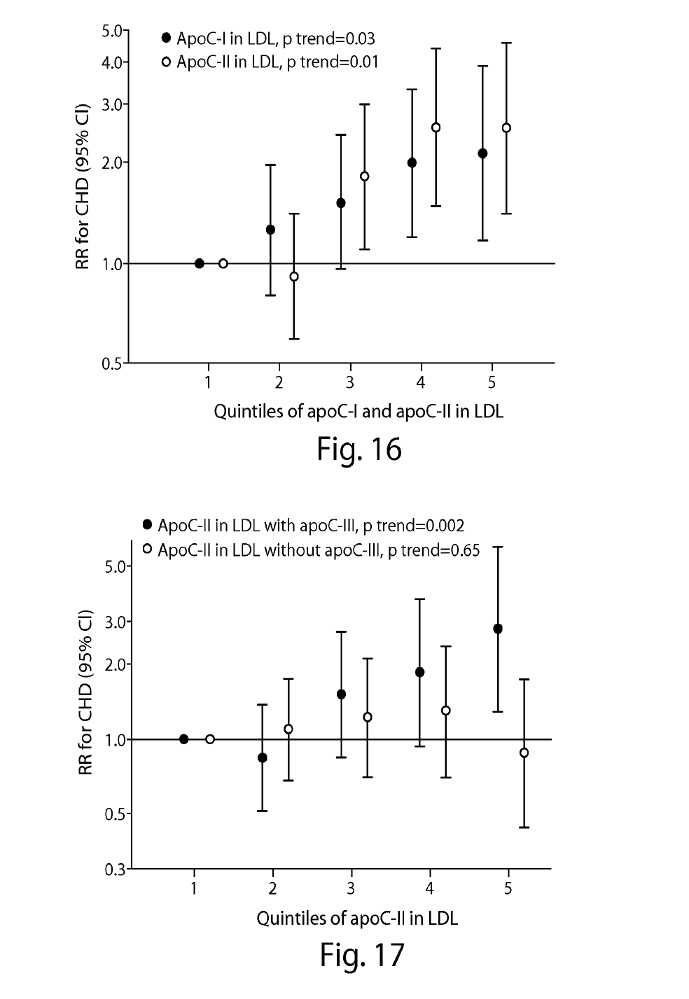

It was observed that both apoC-I and apoC-II in total LDL were associated with risk of CHD (FIG. 16). The elevated RR associated with high levels of apoC-II was attributable to apoC-II in LDL particles that also had apoC-III (FIG. 17). RRs associated with apoC-I in LDL did not differ according to its presence on LDL with or without apoC-III.

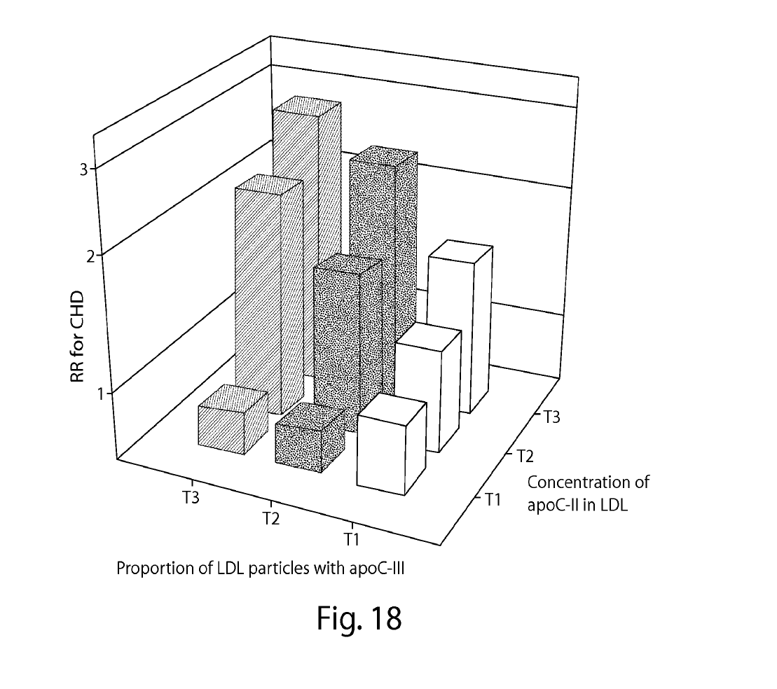

An interaction between apoC-II in LDL and the proportion of the LDL with apoC-III (the most atherogenic lipoprotein) was observed. ApoC-II in LDL was associated with an increased risk of CHD independent of the % of LDL with apoC-III, but individuals in the top tertile of both exposures had the highest RR (2.9; 95% CI:1.6-5.3), compared to those in lowest tertile of both (FIG. 18). The results presented herein demonstrate that the concentration of apoC-II in LDL adds to the cardiovascular risk of LDL with apoC-III.

### Example 5

### Exemplary Atherogenicity Indices

Various atherogenicity indices were generated and evaluated with regard to the power and accuracy of risk assignment.

VLDL & LDL Atherogenicity Index Component Measurements

The indices were comprised of apolipoprotein measurements obtained by laboratory analysis. Whole plasma was fractionated into apoC-III-containing and apoC-III-deficient lipoproteins by immuno-affinity chromatography with goat anti-human apoC-III antibody bound to Sepharose 4B resin (Academy Biomedical, Houston, Tex.). These fractions were then separated into VLDL, IDL+LDL, and HDL by ultracentrifugation with potassium bromide. This resulted in 6 lipoprotein fractions: VLDL with apoC-III, VLDL without apoC-III, LDL with apoC-III, LDL without apoC-III, HDL with apoC-III, and HDL without apoC-III. Measurements of apoA-II, apoB, apoC-I, apoC-II, apoC-III, and apoE were made in these 6 fractions and in whole plasma by sandwich ELISA using polyclonal antibodies (Academy Biomedical, Houston, Tex.) with HRP-OPD detection. Triglyceride and cholesterol were measured by enzymatic assay (Thermo Fisher, Waltham, Mass.).

Calculation of VLDL & LDL Atherogenicity Index Score in Women

The measurements chosen to be factors the index score were those with the greatest predictive power for CHD: apoB in LDL with apoC-III, apoE in LDL with apoC-III, apoCI in LDL with apoC-III, apoCII in apoB lipoproteins, and apoAII in VLDL with apoC-III. The beta-coefficients from the univariate logistic regression model were calculated for the purpose of weighting each component factor score in the overall score calculation. For all factors except apoE in LDL with apoC-III, there was a direct association with CHD risk. These factors were used to calculate component factor scores by first classifying participants into deciles of each factor and assigning the component factor score of 0 for those in the lowest decile (<10th percentile), 1 in the next higher decile, and so on assigning a 9 to those in the highest decile (>90th percentile). Because apoE in LDL with apoC-III was associated with reduced CHD risk, reverse scoring was created. As for the other factors, participants were classified into deciles of apoE in LDL with apoC-III but were assigned the component factor score of 9 for those in the lowest decile (<10th percentile), 8 in the next higher decile, and so on assigning a 0 to those in the highest quintile (>90th percentile).

After calculating component scores for each of the 5 component apolipoprotein measurements, an index score was calculated as the sum of the products of the component scores and their beta-coefficient. Calculated in this way, higher scores are indicative of higher risk. This index score was then modeled using logistic regression to determine the relative risk comparing quintiles 2 through 5 to quintile 1 (lowest score).

Calculation of VLDL & LDL Atherogenicity Index Score in Men

The measurements chosen to be factors the index score were those with the greatest predictive power for CHD: apoB in LDL with apoC-III, TG in LDL, apoCI in LDL, apoCII in LDL, and apoAII in VLDL. The index score was calculated similarly to the score for women. The beta-coefficients from the univariate logistic regression model were calculated for the purpose of weighting each component factor score in the overall score calculation. All factors showed a direct association with CHD risk and so the component factor scores were calculated by first classifying participants into deciles of each factor and assigning the component factor score of 0 for those in the lowest decile (<10th percentile), 1 in the next higher decile, and so on assigning a 9 to those in the highest decile (>90th percentile). After calculating component scores for each of the 5 component apolipoprotein measurements, an index score was calculated as the sum of the products of the component scores and their beta-coefficient. Calculated in this way, higher scores are indicative of higher risk. This index score was then modeled using logistic regression to determine the relative risk comparing quintiles 2 through 5 to quintile 1 (lowest score).

Results

First, the accuracy of risk assignment using the “traditional” risk factors triglycerides (TG) and LDL cholesterol (LDL-C) was evaluated. The index scores were calculated and quintile-ranked for participants of the Nurses' Health Study (NHS) as described in more detail elsewhere herein. Briefly, each participant ID was classified into deciles of “LDL-cholesterol concentration” and a variable was created that indicates in which decile that ID fell. Similarly, each ID was classified into deciles of “triglyceride concentration” and a variable was created that indicates in which decile that ID fell. The “LDL-C” decile variable and the “TG” decile variable were summed into the index score, which, for this index, ranged from 2-20. A statistical analysis was run to compare the relative risk of quintiles with quintile 1 (Q1) as the reference group, assigning Q1 a relative risk of 1. A relative risk higher than 1, accordingly, indicates that an individual within the respective quintile has an elevated risk of CVD as compared to the reference cohort of Q1. The relative risks (RRs) and confidence intervals were calculated for each quintile as follows:

| Quintile | RR | Confidence Interval |
|---|---|---|
| Q1 | 1.00 | |
| Q2 | 1.42 | (0.72, 2.82) |
| Q3 | 2.07 | (1.03, 4.14) |
| Q4 | 1.76 | (0.91, 3.37) |
| Q5 | 2.36 | (1.23, 4.54) |
| TREND RR: | 1.20 | (1.04, 1.38) p = 0.0115 |

The data demonstrate that, while the traditionally assessed factors, LDL-C and TG, provide some predictive power in assigning CVD risk, a better correlation of risk assignment and outcome is desirable.

Various risk indices were developed that provide more powerful predictions than the two-factor analysis above. For example, a five-factor VLDL & LDL Atherogenicity Index in women was developed that included four risk factors and one protective factor:
apoE in LDL with apoC-III (protective)
apoB in LDL with apoC-III (risk)
apoCI in LDL with apoC-III (risk)
apoCII in apoB lipoproteins (risk)
apoAII in VLDL with apoC-III (risk)

Index scores were calculated and quintile-ranked for participants in the Nurses' Health Study (NHS) as described in more detail elsewhere herein. Briefly, each participant ID was classified into deciles of “apoE in LDL with apoC-III (protective),” “apoB in LDL with apoC-III (risk),” “apoCI in LDL with apoC-III (risk),” “apoCII in apoB lipoproteins (risk),” and “apoAII in VLDL with apoC-III (risk),” and a variable was created that indicates in which decile the respective ID fell. Variables for risk factors were inverse from variables for protective factors. The decile variables were summed into the index score, which, for this index, ranged from 5-300. The relative risks (RRs) and confidence intervals were calculated for each quintile as follows:

| Quintile | RR | Confidence Interval |
|---|---|---|
| Q1 | 1.00 | |
| Q2 | 3.00 | (1.39, 6.50) |
| Q3 | 4.03 | (1.86, 8.74) |
| Q4 | 3.81 | (1.71, 8.50) |
| Q5 | 5.42 | (2.34, 12.5) |
| TREND RR: | 1.37 | (1.16, 1.62) p = 0.0003 |

These data demonstrate that the use of an index as provided herein is more powerful in assigning risk than the traditional measurements of LDL cholesterol (LDL-C). It was next evaluated how the accuracy of risk assignment of the index above would be affected by adjusting for the relative risk assigned based on LDL-C measurements. The adjusted relative risk values and confidence intervals are provided below:

| Quintile | RR | Confidence Interval (CI) |
|---|---|---|
| Q1 | 1.00 | |
| Q2 | 2.74 | (1.25, 6.02) |
| Q3 | 3.86 | (1.76, 8.43) |
| Q4 | 3.36 | (1.48, 7.65) |
| Q5 | 4.78 | (2.03, 11.2) |
| TREND RR: | 1.34 | (1.13, 1.60) p = 0.0009 |

Accordingly, the VLDL & LDL Atherogenicity Index in women (NHS) is a more powerful predictor of CVD risk than the two-factor TG and LDL analysis traditionally performed. One important finding of this work is that an assessment based only on the two traditionally assessed parameters, TG and LDL, does not show a trend from Q1-Q5 when scores are adjusted for other known risk factors, e.g., according to the models described in more detail elsewhere herein, while a more differentiated index, e.g. the index provided above, does show such a trend. An exemplary comparison of the two-factor analysis and the five-factor index in such an adjusted model is provided below:

| | |
|---|---|
| Two-factor analysis | Five-factor analysis |

| Q | RR | CI | Q | RR | CI |
|---|---|---|---|---|---|
| Q1 | 1.00 | | Q1 | 1.00 | |
| Q2 | 0.96 | (0.45, 2.04) | Q2 | 2.96 | (1.34, 6.52) |
| Q3 | 1.28 | (0.58, 2.80) | Q3 | 3.98 | (1.73, 9.12) |
| Q4 | 0.93 | (0.42, 2.07) | Q4 | 3.69 | (1.47, 9.28) |
| Q5 | 1.20 | (0.52, 2.75) | Q5 | 5.05 | (1.89, 13.5) |

A seven-factor index score was calculated next, based on combining VLDL & LDL risk factors with HDL protective factors provided herein. The calculation of this score was performed in the same manner as outlined above, assigning each ID a value based on the decile in which it falls for each particular factor, then summing the factors. Protective factors were given reverse values. The factors included the VLDL & LDL Atherogenicity Index in Women (NHS) factors described above as well as an “HDL-cholesterol with apoC-III” and “HDL-cholesterol without apoC-III” protective factors:
apoB in LDL with apoC-III (risk factor)
apoE in LDL with apoC-III (protective factor, reverse decile value)
apoCI in LDL with apoC-III (risk factor)
apoCII in apoB lipoproteins (risk factor)
apoAII in VLDL with apoC-III (risk factor)
HDL-cholesterol with apoC-III (risk factor)
HDL-cholesterol without apoC-III (protective factor, reverse decile value)

Relative Risk values and confidence intervals for the seven-factor score were calculated as follows:

| Quintile | RR | Confidence Interval |
|---|---|---|
| Q1 | 1.00 | |
| Q2 | 3.31 | (1.51, 7.28) |
| Q3 | 3.85 | (1.68, 8.83) |
| Q4 | 4.49 | (1.96, 10.3) |
| Q5 | 6.36 | (2.69, 15.0) |
| TREND RR: | 1.41 | (1.19, 1.68) p = <0.0001 |

The predictive power of this seven-factor score exceeded that of the VLDL & LDL Atherogenicity Index in Women (NHS) index by including the addition of information on protective HDL without apoC-III and dysfunctional non-protective HDL with apoC-III.

Next, a five-factor VLDL & LDL Atherogenicity Index in men was developed based on the research described elsewhere herein. This index included five risk factors:
apoB in LDL with apoC-III
TG in LDL
apoCI in LDL
apoCII in LDL
apoAII in VLDL

Index scores were calculated and quintile-ranked for participants in the Health Professionals Follow-up Study (HPFS) as described in more detail elsewhere herein. The relative risks (RRs) and confidence intervals were calculated for each quintile as follows:

| Quintile | RR | Confidence Interval |
|---|---|---|
| Q1 | 1.00 | |
| Q2 | 1.33 | (0.83, 2.11) |
| Q3 | 1.24 | (0.77, 2.00) |
| Q4 | 1.80 | (1.10, 2.94) |
| Q5 | 3.23 | (1.92, 5.44) |
| TREND RR: | 1.31 | (1.16, 1.47) p = <0.0001 |

These data demonstrate, again, that the use of an index as provided herein is more powerful in assigning risk than the traditional measurements of (LDL-C). It was evaluated for this index as well whether the accuracy of risk assignment could be further improved by adjusting for the relative risk assigned based on LDL-C measurements. The adjusted relative risk values and confidence intervals are provided below:

| Quintile | RR | Confidence Interval |
|---|---|---|
| Q1 | 1.00 | |
| Q2 | 1.34 | (0.83, 2.14) |
| Q3 | 1.20 | (0.73, 1.95) |
| Q4 | 1.64 | (0.98, 2.73) |
| Q5 | 2.83 | (1.65, 4.85) |
| TREND RR: | 1.26 | (1.11, 1.43) p = 0.0003 |

In summary, the data demonstrate that using multi-factor predictive indices as provided herein is more powerful in assessing CVD risk than the traditional methods.

### Example 6

### Exemplary Composite Indices

In one exemplary embodiment, an HDL Protection Index is calculated by combining a classical apolipoprotein index, a thrombogenic index, an inflammation index, and an anti-oxidant index. In one example, the classical apolipoprotein index is calculated based on apoC-III, apoE, and/or apoA-I concentration(s); the thrombogenic index is calculated based on prothrombin, fibrinogen and/or antithrombin-III concentration(s); the inflammation index is calculated based on SAA1, SAA2, and/or beta-2-microglobulin concentration(s); and the anti-oxidant index is based on PON-1 concentration. The indices are calculated in analogy to the calculations described in more detail for the Atherogenicity indices elsewhere herein, and the combined HDL Protection index is constructed by combining the individual indices, e.g., as the sum of the individual indices; as produced by multiplying the individual indices; as produced by mathematical modeling; or as produced by a simple ratio of the individual indices.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any sub-range within the given range as well as any specific value within the stated range in some embodiments, to the tenth of the unit of the lower limit of the range, unless the context clearly dictates otherwise. For example, description of a range from 1 to 6 should be considered to have specifically disclosed subranges such as from 1 to 2, from 1 to 3, from 1 to 4, from 1 to 5, from 2 to 4, from 2 to 6, from 3 to 6 etc., as well as individual numbers to at least the second decimal within that range, for example, 1, 2, 2.7, 3, 4, 5, 5.37, 6, etc.

For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment described or provided herein may be explicitly excluded from any one or more of the claims. Where ranges are given, any value within the range may explicitly be excluded from any one or more of the claims. Any embodiment, element, feature, application, or aspect of the compositions and/or methods disclosed or provided herein, can be excluded from any one or more claims. For purposes of brevity, all of the embodiments in which one or more elements, features, purposes, or aspects is excluded are not set forth explicitly herein.

1. A method of generating an index for assessing risk of a subject having or developing a cardiovascular disease wherein the index is selected from the group consisting of VLDL-LDL Atherogenicity Index, HDL Protection Index, Global Lipoprotein Index, and any combination thereof, the method comprising detecting at least one analyte that is a component of a lipoprotein in a biological sample derived from the subject and determining the concentration of the analyte in the sample to generate an index that is a measure of the risk of the subject for having or developing a cardiovascular disease.

2. A method of generating an index for assessing the risk of a subject for having or developing cardiovascular disease, the method comprising
(i) detecting an analyte that is a component of a lipoprotein in a biological sample derived from the subject; and
(ii) generating an index that is a measure of the risk of the subject for having or developing cardiovascular disease.

3. The method of claim 2, wherein detecting the analyte comprises measuring the presence or a level or concentration of the analyte.

4. The method of claim 3, wherein the level of the analyte is measured via a quantitative or semi-quantitative assay.

5. The method of any one of claims 2-4, wherein generating the index comprises calculating an index score based on the detection and/or quantification of the analyte.

6. The method of any one of claims 2-5, wherein the index generated is an index selected from the group consisting of VLDL-LDL Atherogenicity Index, HDL Protection Index, Global Lipoprotein Index, and any combination thereof.

7. The method of any one of claims 1-6, wherein when the VLDL-LDL Atherogenicity Index value is higher than a control value indicating low risk, there is an increased risk of cardiovascular disease in the subject.

8. The method of claim 1, wherein when the HDL Protection Index value is lower than a control value denoting low risk, there is an increased risk of cardiovascular disease in the subject.

9. The method of claim 1, wherein the VLDL-LDL Atherogenicity Index value is calculated as the sum of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

10. The method of claim 9, wherein when the analyte is associated directly with high risk of cardiovascular disease, a score of 0 is assigned to subjects whose concentrations lie within the first quintile (lowest 20^{th} percentile), a score of 1 is assigned to subjects whose concentrations lie within the second quintile, a score of 2 is assigned to subjects whose concentrations lie within third quintile, a score of 3 is assigned to subjects whose concentrations lie within fourth quintile, and a score of 4 is assigned to subjects whose concentrations lie within the fifth quintile (highest 20^{th} percentile).

11. The method of claim 10, wherein the analyte associated with cardiovascular disease is selected from the group consisting of apoC-II, apoC-III, and any combination thereof.

12. The method of claim 9, wherein when the analyte is associated with protection against cardiovascular disease, a score of 4 is assigned to subjects whose concentrations lie within the first quintile (lowest 20^{th} percentile), a score of 3 assigned to subjects whose concentrations lie within the second quintile, a score of 2 assigned to subjects whose concentrations lie within third quintile, a score of 1 assigned to subjects whose concentrations lie within fourth quintile, and a score of 0 assigned to subjects whose concentrations lie within the fifth quintile (highest 20^{th} percentile).

13. The method of claim 12, wherein the analyte associated with protection against cardiovascular disease is apoE.

14. The method of claim 1, wherein the VLDL-LDL Atherogenicity Index value is calculated as the sum of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

15. The method of claim 1, wherein the VLDL-LDL Atherogenicity Index value is calculated as the multiplication of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

16. The method of claim 15, wherein when the analyte is associated directly with cardiovascular disease, a score of 1 is assigned to subjects whose concentrations lie within the first quintile (lowest 20^{th} percentile), a score of 2 is assigned to subjects whose concentrations lie within the second quintile, a score of 3 is assigned to subjects whose concentrations lie within third quintile, a score of 4 is assigned to subjects whose concentrations lie within fourth quintile, and a score of 5 is assigned to subjects whose concentrations lie within the fifth quintile (highest 20^{th} percentile).

17. The method of claim 16, wherein the analyte associated with cardiovascular disease is selected from the group consisting of apoC-II, apoC-III, and any combination thereof.

18. The method of claim 15, wherein when the analyte is associated with protection against cardiovascular disease, a score of 1 is assigned to subjects whose concentrations lie within the first quintile (lowest 20^{th} percentile), a score of 0.8 is assigned to subjects whose concentrations lie within the second quintile, a score of 0.6 is assigned to subjects whose concentrations lie within third quintile, a score of 0.4 is assigned to subjects whose concentrations lie within fourth quintile, and a score of 0.2 assigned to subjects whose concentrations lie within the fifth quintile (highest 20^{th} percentile).

19. The method of claim 18, wherein the analyte associated with protection against cardiovascular disease is apoE.

20. The method of claim 1, wherein the VLDL-LDL Atherogenicity Index value is calculated as the multiplication of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

21. The method of claim 1, wherein the VLDL-LDL Atherogenicity Index value is calculated as the sum of scores of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

22. The method of claim 21, wherein the score is calculated as the product of the coefficient from the linear regression of the analyte on CHD risk and the concentration of the analyte.

23. The method of claim 22, wherein the analyte is selected from the group consisting of apoC-II, apoC-III, and apoE in apoB-lipoproteins, and any combination thereof.

24. The method of claim 1, wherein the HDL Protection Index value is calculated as the summation of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

25. The method of claim 24, wherein when the analyte is associated directly with a higher rate of cardiovascular disease, a score of 4 is assigned to subjects whose concentrations lie within the first quintile (lowest 20^{th} percentile), a score of 3 is assigned to subjects whose concentrations lie within the second quintile, a score of 2 is assigned to subjects whose concentrations lie within third quintile, a score of 1 is assigned to subjects whose concentrations lie within fourth quintile, and a score of 0 is assigned to subjects whose concentrations lie within the fifth quintile (highest 20^{th} percentile).

26. The method of claim 25, wherein the analyte associated with cardiovascular disease is apoC-III in HDL and apoE in HDL.

27. The method of claim 24, wherein when the analyte is associated with protection against cardiovascular disease, a score of 0 assigned to subjects whose concentrations lie within the first quintile (lowest 20^{th} percentile), a score of 1 assigned to subjects whose concentrations lie within the second quintile, a score of 2 assigned to subjects whose concentrations lie within third quintile, a score of 3 assigned to subjects whose concentrations lie within fourth quintile, and a score of 4 assigned to subjects whose concentrations lie within the fifth quintile (highest 20^{th} percentile).

28. The method of claim 27, wherein the analyte associated with protection against cardiovascular disease is HDL without one or more of apoC-III and apoE.

29. The method of claim 1, wherein the HDL Protection Index value is calculated as the sum of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

30. The method of claim 1, wherein the HDL Protection Index value is calculated as the multiplication of scores calculated based on population distributions of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

31. The method of claim 30, wherein when the analyte is associated directly with cardiovascular disease, a score of 0.2 is assigned to subjects whose concentrations lie within the first quintile (lowest 20^{th} percentile), a score of 0.4 is assigned to subjects whose concentrations lie within the second quintile, a score of 0.6 is assigned to subjects whose concentrations lie within third quintile, a score of 0.8 is assigned to subjects whose concentrations lie within fourth quintile, and a score of 1 is assigned to subjects whose concentrations lie within the fifth quintile (highest 20^{th} percentile).

32. The method of claim 31, wherein the analyte associated with cardiovascular disease is apoC-III in HDL and apoE in HDL.

33. The method of claim 30, wherein when the analyte is associated with protection against cardiovascular disease, a score of 1 is assigned to subjects whose concentrations lie within the first quintile (lowest 20^{th} percentile), a score of 2 assigned to subjects whose concentrations lie within the second quintile, a score of 3 assigned to subjects whose concentrations lie within third quintile, a score of 4 assigned to subjects whose concentrations lie within fourth quintile, and a score of 5 assigned to subjects whose concentrations lie within the fifth quintile (highest 20^{th} percentile).

34. The method of claim 33, wherein the analyte associated with protection against cardiovascular disease is HDL without one or more of apoC-III and apoE.

35. The method of claim 1, wherein the HDL Protection Index value is calculated as the multiplication of scores calculated based on relative risks of cardiovascular disease calculated from epidemiological studies for at least one analyte that is a component of a lipoprotein, wherein the analyte is selected from the group consisting of an integral apolipoprotein, a non-integral apolipoprotein, a lipoprotein-associated protein listed in Table 1, and any combination thereof.

36. The method of claim 1, wherein the HDL Protection Index value is calculated as the sum of scores of at least one analyte that is a component of a lipoprotein, wherein the analyte is selected from the group consisting of an integral apolipoprotein, a non-integral apolipoprotein, a lipoprotein-associated protein listed in Table 1, and any combination thereof.

37. The method of claim 36, wherein the score is calculated as the product of the coefficient from the linear regression of the analyte on CHD risk and the concentration of the analyte.

38. The method of claim 37, wherein the analyte is selected from the group consisting of apoC-III in HDL, apoE in HDL, apoAI without apoC-III or apoE, and any combination thereof.

39. The method of claim 1, wherein the analyte is selected from the group consisting of an integral apolipoprotein, a non-integral apolipoprotein, a lipoprotein-associated protein listed in Table 1, and any combination thereof.

40. The method of claim 39, wherein the integral apolipoprotein is selected from the group consisting of apoA-I, apoB, and any combination thereof.

41. The method of claim 39, wherein the non-integral apolipoprotein is selected from the group consisting of apoA-II, apoC-I, apoC-II, apoC-III, apoE, and any combination thereof.

42. The method of claim 39, wherein the lipoprotein is selected from the group consisting of VLDL, LDL, HDL, and any combination thereof.

43. The method of claim 42, wherein the lipoprotein is computed as the cholesterol or triglyceride concentration.

44. The method of any of claim 1, or 6-43, wherein the Global Lipoprotein Index is constructed by combining the VLDL-LDL Atherogenicity Index and the HDL Protection Index.

45. The method of claim 44, wherein the Global Lipoprotein Index is constructed as the sum of the VLDL-LDL Atherogenicity Index and the HDL Protection Index; as produced by multiplying the VLDL-LDL Atherogenicity Index and the HDL Protection Index; as produced by mathematical modeling; or as produced by a simple ratio of the VLDL-LDL Atherogenicity Index and the HDL Protection Index.

46. A method of assessing a cardiovascular disease in a subject, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease.

47. A method of selecting a subject for participation in a clinical trial, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to select a subject for participation in a clinical trial.

48. A method of assessing the efficacy of a pharmaceutical agent, dietary supplement or food product in preventing or treating a cardiovascular disease in a subject in need thereof, the method comprising generating an index for assessing risk of developing a cardiovascular disease wherein the index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to assess the efficacy of a pharmaceutical agent in treating a subject in need thereof.

49. A method of assessing the efficacy of a therapeutic agent, such as a pharmaceutical agent, dietary supplement or food product in treating a cardiovascular disease in a subject in need thereof, the method comprising generating an index that is itself a target for the pharmaceutical agent, dietary supplement or food product. The index is selected from the group consisting of VLDL-LDL Atherogenicity Index, HDL Protection Index, Global Lipoprotein Index, and any combination thereof, by detecting at least one analyte that is a component of a lipoprotein in a biological sample derived from the subject and determining the concentration of the analyte in the sample to generate an index that is a measure of risk of the subject having or developing a cardiovascular disease in order to assess the efficacy of a therapeutic agent in treating a subject in need thereof. The therapeutic agent seeks to lower the VLDL-LDL Atherogenicity Index, raise the HDL Protection Index, and/or lower the Global Lipoprotein Index, thereby lowering the risk of cardiovascular disease in the subject or group of subjects.

50. A method of generating an HDL Protection Index, the method comprising combining at least two indices from the group consisting of a classical apolipoprotein index, a thrombogenic index, an inflammation index, and an anti-oxidant index.
